SEQUENCE LISTING (91 sequences)

SEQ ID NO: 1 - DNA, 24 bp, Artificial Sequence
  HMGCR exon1 probe
attgtgtgtgggaccgtaatggct

SEQ ID NO: 2 - DNA, 17 bp, Artificial Sequence
  primer
aaatgcccggcagcttg

SEQ ID NO: 3 - DNA, 20 bp, Artificial Sequence
  primer
ctgccaatgctgccataagt

SEQ ID NO: 4 - DNA, 27 bp, Artificial Sequence
  PYK1 promoter probe
cgaatatcgttttgatggcgagccttt

SEQ ID NO: 5 - DNA, 26 bp, Artificial Sequence
  primer
agaggttcttggaaatgaaaagttac

SEQ ID NO: 6 - DNA, 23 bp, Artificial Sequence
  primer
ccttcccatatgatgctaggtac

SEQ ID NO: 7 - DNA, 27 bp, Artificial Sequence
  PYK1 5′ probe
agaattgtacccaggtagaccattggc

SEQ ID NO: 8 - DNA, 21 bp, Artificial Sequence
  primer
ttctgatttctggacccttgg

SEQ ID NO: 9 - DNA, 20 bp, Artificial Sequence
  primer
cattgacaacgccagaagtc

SEQ ID NO: 10 - DNA, 28 bp, Artificial Sequence
  PYK1 3′ probe
ctctcacttgtacagaggtgtcttggga

SEQ ID NO: 11 - DNA, 21 bp, Artificial Sequence
  primer
tcagagacaggttccttttcg

SEQ ID NO: 12 - DNA, 19 bp, Artificial Sequence
  primer
ttaccagatgcccaagagc

SEQ ID NO: 13 - DNA, 25 bp, Artificial Sequence
  PMA1 promoter probe
ccgcttatgctcccctccattagtt

SEQ ID NO: 14 - DNA, 19 bp, Artificial Sequence
  primer
tgacgaaacgtggtcgatg

SEQ ID NO: 15 - DNA, 30 bp, Artificial Sequence
  primer
aaattagatgttagacgataatgataggac

SEQ ID NO: 16 - DNA, 20 bp, Artificial Sequence
  PMA1 5′ probe
ttgccgccggtgaagctaga

SEQ ID NO: 17 - DNA, 22 bp, Artificial Sequence
  primer
gacagtgataacgatggtccag

SEQ ID NO: 18 - DNA, 21 bp, Artificial Sequence
  primer
accgtaagatgggtcagtttg

SEQ ID NO: 19 - DNA, 28 bp, Artificial Sequence
  PMA1 3′ probe
cgtgtctggatctggtctatcggtatct

SEQ ID NO: 20 - DNA, 24 bp, Artificial Sequence
  primer
actggactgatattgttactgtcg

SEQ ID NO: 21 - DNA, 23 bp, Artificial Sequence
  primer
catcaatctgtcaaaggcttcag

SEQ ID NO: 22 - DNA, 26 bp, Artificial Sequence
  MDH2 promoter probe
aaagtcaaatcggaccttcccaacct

SEQ ID NO: 23 - DNA, 24 bp, Artificial Sequence
  primer
tttgtttagtggcattttgtgttg

SEQ ID NO: 24 - DNA, 23 bp, Artificial Sequence
  primer
ccttagataccttccgtagttcc

SEQ ID NO: 25 - DNA, 23 bp, Artificial Sequence
  MDH2 5′ probe
tggtatcgggcagtcgttatcgc

SEQ ID NO: 26 - DNA, 22 bp, Artificial Sequence
  primer
ctggtattgcaactgagctttc

SEQ ID NO: 27 - DNA, 20 bp, Artificial Sequence
  primer
attgccattttaggtgctgc

SEQ ID NO: 28 - DNA, 24 bp, Artificial Sequence
  MDH2 3′ probe
attattgcctctggtggacggtgc

SEQ ID NO: 29 - DNA, 19 bp, Artificial Sequence
  primer
aacttccccattgctcctg

SEQ ID NO: 30 - DNA, 19 bp, Artificial Sequence
  primer
gttgcgttccatgtcgttc

SEQ ID NO: 31 - DNA, 25 bp, Artificial Sequence
  HMG1 promoter probe
agtgcgaagaaaacgtagtgcgagt

SEQ ID NO: 32 - DNA, 22 bp, Artificial Sequence
  primer
gaagagttggaagaccttcagc

SEQ ID NO: 33 - DNA, 23 bp, Artificial Sequence
  primer
agatcctatagctagtacggacc

SEQ ID NO: 34 - DNA, 28 bp, Artificial Sequence
  HMG1 5′ probe
tgaatgacggatagataagcgaatgcgg

SEQ ID NO: 35 - DNA, 18 bp, Artificial Sequence
  primer
cggcgaaacgaccaattc

SEQ ID NO: 36 - DNA, 23 bp, Artificial Sequence
  primer
gtgttggagtctttatttggagc

SEQ ID NO: 37 - DNA, 26 bp, Artificial Sequence
  HMG1 3′ probe
aggttttgttggttcagcaggtttcc

SEQ ID NO: 38 - DNA, 22 bp, Artificial Sequence
  primer
acgatttatatcagtggcgtcc

SEQ ID NO: 39 - DNA, 20 bp, Artificial Sequence
  primer
cggccatttggttcaaagtc

SEQ ID NO: 40 - DNA, 28 bp, Artificial Sequence
  ERG11 promoter probe
tcgttaactcgtggagatgcacaatagg

SEQ ID NO: 41 - DNA, 22 bp, Artificial Sequence
  primer
gcaggagacatcgattttatgc

SEQ ID NO: 42 - DNA, 21 bp, Artificial Sequence
  primer
gaggcttttcgaatacatgcg

SEQ ID NO: 43 - DNA, 26 bp, Artificial Sequence
  ERG11 5′ probe
tgggccaatggtaaagccaagaaatg

SEQ ID NO: 44 - DNA, 21 bp, Artificial Sequence
  primer
ttggagaggcattggaatacg

SEQ ID NO: 45 - DNA, 28 bp, Artificial Sequence
  primer
tcctttctcaaagaatatagtaattgcc

SEQ ID NO: 46 - DNA, 27 bp, Artificial Sequence
  ERG11 3′ probe
accgttccacctcctgactttacatct

SEQ ID NO: 47 - DNA, 20 bp, Artificial Sequence
  primer
aaatggcattacccagaggg

SEQ ID NO: 48 - DNA, 24 bp, Artificial Sequence
  primer
tttgttctggatttctcttttccc

SEQ ID NO: 49 - DNA, 28 bp, Artificial Sequence
  HMG2 promoter probe
ccctttggtactccacctcttcaagatg

SEQ ID NO: 50 - DNA, 21 bp, Artificial Sequence
  primer
cacctcaagaacctaccacac

SEQ ID NO: 51 - DNA, 24 bp, Artificial Sequence
  primer
agtgtccttactataacttgctcg

SEQ ID NO: 52 - DNA, 24 bp, Artificial Sequence
  HMG2 5′ probe
ctcagctctaaagaagccgccgat

SEQ ID NO: 53 - DNA, 22 bp, Artificial Sequence
  primer
ggtctcctgtgtctgatacatg

SEQ ID NO: 54 - DNA, 19 bp, Artificial Sequence
  primer
tcaagggaaggcaaagtcg

SEQ ID NO: 55 - DNA, 23 bp, Artificial Sequence
  HMG2 3′ probe
ccggtcacctggtacaaagccat

SEQ ID NO: 56 - DNA, 20 bp, Artificial Sequence
  primer
gtgaactgtctctgtgctcc

SEQ ID NO: 57 - DNA, 21 bp, Artificial Sequence
  primer
gccctttgttacttggttgtg

SEQ ID NO: 58 - DNA, 25 bp, Artificial Sequence
  primer
tgccatgtatgcaactgctctacca

SEQ ID NO: 59 - DNA, 20 bp, Artificial Sequence
  primer
acacccgtctttgatgctag

SEQ ID NO: 60 - DNA, 22 bp, Artificial Sequence
  primer
atgttctcacgctctgttctag

SEQ ID NO: 61 - DNA, 26 bp, Artificial Sequence
  YFR057W promoter probe
acaagggaacaatgagcagagggaaa

SEQ ID NO: 62 - DNA, 23 bp, Artificial Sequence
  primer
caaacaagtaggaatgcgaaagg

SEQ ID NO: 63 - DNA, 21 bp, Artificial Sequence
  primer
aagtgctaaaggaatccccag

SEQ ID NO: 64 - DNA, 19 bp, Artificial Sequence
  YFR057W 5′ probe
tgctcggccaagtcaagcg

SEQ ID NO: 65 - DNA, 20 bp, Artificial Sequence
  primer
gcttggcggtgtctttaatg

SEQ ID NO: 66 - DNA, 28 bp, Artificial Sequence
  primer
tgatatttggacctactagtgtctatag

SEQ ID NO: 67 - DNA, 28 bp, Artificial Sequence
  YFR057W 3′ probe
tgtcaaaggaattttttgcgcctagtgc

SEQ ID NO: 68 - DNA, 23 bp, Artificial Sequence
  primer
aaaggattttagcaacgacttcg

SEQ ID NO: 69 - DNA, 26 bp, Artificial Sequence
  primer
ttgtcgaatcttccagtaaatttctc

SEQ ID NO: 70 - DNA, 28 bp, Artificial Sequence
  YFR057W  3′-UTR probe
actttctggaatagcgttcggaatgtgt

SEQ ID NO: 71 - DNA, 21 bp, Artificial Sequence
  primer
cactcgttaggatcacgttcg

SEQ ID NO: 72 - DNA, 20 bp, Artificial Sequence
  primer
tgtcggagagttaacaagcg

SEQ ID NO: 73 - DNA, 24 bp, Artificial Sequence
  ARS504 probe
ttggtcttccctacgcatttcccc

SEQ ID NO: 74 - DNA, 22 bp, Artificial Sequence
  primer
gccgtttcaatctatcgtcatg

SEQ ID NO: 75 - DNA, 21 bp, Artificial Sequence
  primer
atccagctgactcatttcctg

SEQ ID NO: 76 - DNA, 19 bp, Artificial Sequence
  primer
tttcggcccgttttatcgt

SEQ ID NO: 77 - DNA, 20 bp, Artificial Sequence
  primer
gagccaccatgcttctcatt

SEQ ID NO: 78 - DNA, 20 bp, Artificial Sequence
  primer
tgggggttgcttgttatgtg

SEQ ID NO: 79 - DNA, 20 bp, Artificial Sequence
  primer
aacagcgagagatcgacaga

SEQ ID NO: 80 - DNA, 23 bp, Artificial Sequence
  primer
aatacagtgtctgatcggaggat

SEQ ID NO: 81 - DNA, 21 bp, Artificial Sequence
  primer
gcggaattgggatttttacct

SEQ ID NO: 82 - DNA, 20 bp, Artificial Sequence
  primer
gaatagtccctcggggttga

SEQ ID NO: 83 - DNA, 22 bp, Artificial Sequence
  primer
tcgtgttggaatcagttgatga

SEQ ID NO: 84 - DNA, 20 bp, Artificial Sequence
  primer
cgaagcgtttgatcgggtat

SEQ ID NO: 85 - DNA, 21 bp, Artificial Sequence
  primer
ttcgactgtttaccaactgga

SEQ ID NO: 86 - DNA, 21 bp, Artificial Sequence
  primer
cttggaacttcccgagagctt

SEQ ID NO: 87 - DNA, 22 bp, Artificial Sequence
  primer
caccacttcacaaacccaccta

SEQ ID NO: 88 - DNA, 21 bp, Artificial Sequence
  primer
cccatgattgacagtcgttga

SEQ ID NO: 89 - DNA, 20 bp, Artificial Sequence
  primer
agggctatgagttgccaaga

SEQ ID NO: 90 - DNA, 20 bp, Artificial Sequence
  primer
acaccttcgagacttccctt

SEQ ID NO: 91 - DNA, 20 bp, Artificial Sequence
  primer
tgtacagcggcgttaaaaca

(19) United States
(12) Patent Application Publication
(10) Pub. No.: US 2016/0168622 A1
(43) Pub. Date: Jun. 16, 2016

(54) IMMEDIATE CHROMATIN IMMUNOPRECIPITATION AND ANALYSIS

(71) Applicant: Purdue Research Foundation, West Lafayette, IN (US)

(72) Inventors: Scott Douglas Briggs, West Lafayette, IN (US); Kayla Marie Harmeyer, Philadelphia, PA (US); Paul Francis South, Champaign, IL (US)

(21) Appl. No.: 14/965,732

(22) Filed: Dec. 10, 2015

Related U.S. Application Data
(60) Provisional application No. 62/090,087, filed on Dec. 10, 2014.

(51) Int. Cl. C12Q 1/68; G01N 33/53

(52) U.S. Cl. CPC C12Q 1/6804; G01N 33/5308

(57) ABSTRACT

The present invention relates to a newly developed immediate chromatin immunoprecipitation procedure (“ZipChIP”). ZipChIP significantly reduces the time and increases sensitivity allowing for rapid screening of multiple loci. ZipChIP enables the detection of histone modifications (e.g., H3K4 mono- and trimethylation) and at least two yeast histone demethylases, Jhd2 and Rph1, which were previously found difficult to detect using standard methods. ZipChIP further relates to the enrichment of the histone deacetylase Sir2 at heterochromatin in yeast and enrichment of the chromatin remodeler, PICKLE, in Arabidopsis thaliana.

### CROSS REFERENCE TO RELATED APPLICATIONS

This application claims the benefit of U.S. Provisional Patent Application No. 62/090,087 filed on Dec. 10, 2014 the disclosure of which is incorporated herein by reference in its entirety.

### GOVERNMENT SUPPORT CLAUSE

This invention was made with government support under 0918954 awarded by the National Science Foundation. The government has certain rights in the invention.

### TECHNICAL FIELD

The present disclosure generally relates to analyzing DNA and RNA, and in particular to novel methods and/or buffer systems to extract chromatin and analyze the associated nucleic acids while keeping the chromatin conjugated to a solid support.

### BACKGROUND AND SUMMARY

This section introduces aspects that may help facilitate a better understanding of the disclosure. Accordingly, these statements are to be read in this light and are not to be understood as admissions about what is or is not prior art.

Characterizing the dynamics of the chromatin structure and associated proteins is fundamentally important in the understanding of cellular growth and differentiation. Chromatin is the protein-DNA complex that packages DNA in the nucleus of eukaryotic cells. The basic unit of chromatin is the nucleosome, which is composed of 146 base pairs of DNA wrapped around an octamer of histone proteins. Changing how the DNA is packaged in the nucleus affects many DNA template processes, including gene transcription and DNA repair and replication. As a result, detailed analysis of how chromatin features change in time and space is critical in understanding how these processes are regulated. Changes in DNA packaging can be modulated by a variety of chromatin associated proteins, such as transcription factors and remodelers, and also by posttranslational modifications (PTMs) on histones. The development of chromatin immunoprecipitation (ChIP), ChIP-chip, and ChIP-seq has led to a significant increase in the amount of information gained about the nature of chromatin in respect to the localization of histone variants and of chromatin associated proteins in the genome. Unfortunately, standard ChIP methods are time consuming, expensive, quite laborious, and are subject to high rates of experimental error due to the large number of steps involved. Standard ChIP analysis typically takes 3-4 days, from growing cultures to data analysis. To reduce the time of ChIP analysis, other groups have developed “Fast” ChIP protocols that can significantly reduce the time to complete an experiment. One reported fast ChIP protocol showed that incubation of antibody with chromatin in an ultrasonic bath significantly reduces incubation time. This protocol also included treating the precipitated chromatin at 100° C. with chelex-100 resin to extract the DNA from proteins. This approach can be used in both standard PCR and in quantitative real-time PCR (qRT-PCR), but the 15 minute incubation time in an ultrasonic bath may not be sufficient for some antibody-epitope interactions, which would significantly decrease the sensitivity of the ChIP procedure. In addition, the use of chelex-100 resin not only leads to additional time and cost, but contamination of a PCR reaction with chelex-100 resin prevents DNA amplification. There exists a need for a method which significantly reduces the time and increases sensitivity allowing for rapid screening of multiple loci within nucleic acid sequences.

Some first embodiments of the present disclosure, include a methods of analyzing chromatin, comprising; conjugating a portion of solubilized chromatin with at least one antibody, wherein the antibody is attached to a water insoluble support, to form a chromatin water insoluble support complex, immunoprecipitating the chromatin water insoluble support complex, to form a immunopreciptated chromatin water insoluble support complex; and analyzing the immunopreciptated chromatin water insoluble support complex for the presence of chromatin.

Some second embodiments include the methods of the first embodiment, wherein the water insoluble support complex, is at least one structure selected from the group consisting of: a metal bead, an agarose bead, a sepharose, or a chip surface.

Some third embodiments includes the methods of any of the first and/or the second embodiments, wherein the solubilized chromatin is extracted from at least one cell selected from the group of prokaryotic cells, eukaryotic cell, bacteria cells, plant cells, fungal cells, and animal cells.

Some fourth embodiments include the methods of any of the first to the third embodiments, wherein the solubilized chromatin is extracted from at least one cell using a SB-140 lysis buffer.

Some fifth embodiments include the method of any of the first to the fourth embodiments, wherein the antibody is capable or preferentially binding to at least one post translationally modified protein.

Some sixth embodiments include the methods of any of the first to the fifth embodiments, wherein the antibody is capable or preferentially binding to at least one methylated protein.

Some seventh embodiments include the method of any of the first to the sixth embodiments, further including the step of washing the immunoprecipitating the chromatin water insoluble support complex.

Some eighth embodiments include the methods of any of the first to the seventh embodiments, wherein the analytical step includes the use of DNA primers.

Some ninth embodiments include methods of analyzing nucleic acids, comprising: conjugating a portion of solubilized chromatin with at least one antibody, wherein the antibody is attached to a water insoluble support, to form a chromatin water insoluble support complex; immunoprecipitating the chromatin water insoluble support complex, to form a immunopreciptated chromatin water insoluble support complex; and analyzing the immunopreciptated chromatin water insoluble support complex for the presence DNA and/or RNA.

Some tenth embodiments include the methods of ninth embodiment, wherein the water insoluble support complex, is at least one structure selected from the group consisting of: a metal bead, an agarose bead, a sepharose, or a chip surface.

Some eleventh embodiments include the methods of any of the ninth to the tenth embodiments, wherein the solubilized chromatin is extracted from at least one cell selected from the group of prokaryotic cells, eukaryotic cell, bacteria cells, plant cells, fungal cells, and animal cells.

Some twelfth embodiments include the methods of any of the ninth to the eleventh embodiments, wherein the solubilized chromatin is extracted from at least one cell using a SB-140 lysis buffer.

Some thirteenth embodiments include the methods of any of the ninth to the twelfth embodiments, wherein the antibody is capable or preferentially binding to at least one post translationally modified protein.

Some fourteenth embodiments include the methods of any of the ninth to the thirteenth embodiments, wherein the antibody is capable or preferentially binding to at least one methylated protein.

Some fifteenth embodiments include the methods of any of the ninth to the fourteenth embodiments, further including the step of washing the immunoprecipitating the chromatin water insoluble support complex.

Some sixteenth embodiments include the methods of any of the ninth to the fifteenth embodiments, wherein the analytical step includes the use of nucleic acid primers.

Some seventeenth embodiments includes at least one kit, comprising at least one first antibody attached to at least one water insoluble support, wherein the antibody preferentially binds to at least one protein present in a portion of chromatin to form a chromatin antibody complex; and at least one lysate buffer, wherein said buffer is suitable for forming water soluble chromatin.

Some eighteenth embodiments includes at least one of the kits of the seventeenth embodiment, further including a wash buffer, wherein the wash buffer is suitable for washing said chromatin antibody complex.

Some nineteenth embodiments includes a kit of any of the seventeenth to the eighteenth embodiments, further including at least one primer, that at least one primer binds to at least one portion of a nucleic acid present in said chromatin antibody complex.

Some twentieth embodiments includes at least one of the kits of any of the seventeenth to the nineteenth embodiments, wherein the water insoluble support is at least structure selected from the group consisting of metal, agarose, or sepharose beads.

A twenty first embodiment of the present disclosure includes a method of analyzing chromatin comprising the steps of: providing solubilized chromatin and a plurality of beads having at least one antibody bound to individual beads, conjugating the solubilized chromatin to the at least one antibody and generating a chromatin-antibody-bead complex, immunoprecipitating the chromatin-bead complex, washing the chromatin-antibody-bead complex with SB-140 wash buffer, SB-500 wash buffer, and 1×PBS at least once with each, resuspending the chromatin-antibody-bead complex in water, and performing polymerase chain reaction on the chromatin-antibody-bead complex.

A twenty second embodiment includes the method of the twenty first embodiment, wherein the plurality of beads are magnetic.

A twenty third embodiment includes the method of any of the twenty first to the twenty second embodiments, wherein the solubilized chromatin was extracted from a cell using SB-140 lysis buffer.

A twenty fourth embodiment includes the method of any of the twenty first to the twenty third embodiments, wherein the cell is a eukaryotic cell, a plant cell, a mammalian cell, or a prokaryotic cell.

A twenty fifth embodiment includes the method of any of the twenty first to the twenty fourth embodiments, wherein the bead is made of agarose.

A twenty sixth embodiment includes the method of any of the twenty first to the twenty fifth embodiments, wherein the at least one antibody is able to identify methylated proteins.

A twenty seventh embodiment includes the method of any of the twenty first to the twenty sixth embodiments, wherein the at least one antibody is able to identify proteins or posttranslationally modified proteins.

A twenty eighth embodiment includes the method of any of the twenty first to the twenty seventh embodiments, wherein there is a linker between at least one antibody and the bead.

A twenty ninth embodiment includes the method of any of the twenty first to the twenty eighth embodiments, further including analyzing the results of the polymerase chain reaction or a quantitative-real time PCR.

A thirtieth embodiment includes the method of any of the twenty first to the twenty ninth embodiments, wherein further including heating the bound chromatin or DNA and eluting it from the bead.

A thirty first embodiment includes the method of any of the twenty first to the thirtieth embodiments, wherein the solubilized chromatin includes DNA and/or RNA.

A thirty second embodiment includes a method of analyzing nucleic acids comprising the steps of: providing solubilized chromatin and a plurality of beads having at least one binding molecule bound to individual beads, conjugating the solubilized chromatin to the at least one binding molecule and generating a chromatin-molecule-bead complex, immunoprecipitating the chromatin-molecule-bead complex, washing the chromatin-antibody-bead complex with a first wash buffer, a second wash buffer, and 1×PBS at least once with each, resuspending the chromatin-molecule-bead complex, and performing analysis on the chromatin-molecule-bead complex.

A thirty third embodiment includes the method of the thirty second embodiment, wherein the wash buffers include 0.1% Triton X-100.

A thirty fourth embodiment includes the method of the thirty second to the thirty third embodiments, wherein the solubilized chromatin includes DNA and/or RNA.

A thirty fifth embodiment includes the method of the thirty second to the thirty fourth embodiments, wherein the bead is metal, agarose, sepharose, or magnetic.

A thirty sixth embodiment includes the method of thirty second to the thirty fifth embodiments, wherein the binding molecule is bound indirectly through a linker.

A thirty seventh embodiment includes the method of thirty second to the thirty sixth embodiments, wherein the binding molecule is chosen from a peptide, protein, ligand, small molecule, DNA sequence, RNA sequence, a fragment thereof, or a combination thereof.

### BRIEF DESCRIPTION OF THE FIGURES

FIG. 1A. Flow diagram depicting the steps of ZipChIP starting at soluble chromatin to samples ready for qRT-PCR analysis.

FIG. 1B. Graphs illustrating relative levels of H3K4 trimethylation (H3K4me3) resulted from ZipChIP and standard ChIP methods where PrimeTime qPCR probes were targeted toward the promoter (P), 5′-ORF (5), and 3′-ORF (3) of an actively transcribed gene PYK1.

FIG. 1C. Graphs illustrating relative levels of H3K4 trimethylation (H3K4me3) resulted from ZipChIP and standard ChIP methods where PrimeTime qPCR probes were targeted toward the promoter (P), 5′-ORF (5), and 3′-ORF (3) of an actively transcribed gene PMA1.

FIG. 1D. Graphs illustrating relative levels of H3K4 trimethylation (H3K4me3) resulted from ZipChIP and standard ChIP methods where PrimeTime qPCR probes were targeted toward the promoter (P), 5′-ORF (5), and 3′-ORF (3) of a sub-telomere gene YFRO57W.

FIG. 2A. Graphs illustrating relative levels of Jhd2-3×FLAG resulted from ZipChIP and standard ChIP methods that were performed on BY4741 WT strain and a JHD2-3×FLAG strain with the tag integrated at JHD2's endogenous locus where PrimeTime qPCR probes were targeted toward the promoter (P), 5′-ORF (5), and 3′-ORF (3) of an actively transcribed gene PYK1.

FIG. 2B. Graphs illustrating relative levels of Jhd2-3×FLAG resulted from ZipChIP and standard ChIP methods that were performed on BY4741 WT strain and a JHD2-3×FLAG strain with the tag integrated at JHD2's endogenous locus where PrimeTime qPCR probes were targeted toward the promoter (P), 5′-ORF (5), and 3′-ORF (3) of an actively transcribed gene PMA1.

FIG. 2C. Graphs illustrating relative levels of Jhd2-3×FLAG resulted from ZipChIP and standard ChIP methods that were performed on BY4741 WT strain and a RPH1-3×FLAG strain with the tag integrated at RPH1's endogenous locus where PrimeTime qPCR probes were targeted toward the promoter (P), 5′-ORF (5), and 3′-ORF (3) of an actively transcribed gene PYK1.

FIG. 2D. Graphs illustrating relative levels of Jhd2-3×FLAG resulted from ZipChIP and standard ChIP methods that were performed on BY4741 WT strain and a RPH1-3×FLAG strain with the tag integrated at RPH1's endogenous locus where PrimeTime qPCR probes were targeted toward the promoter (P), 5′-ORF (5), and 3′-ORF (3) of an actively transcribed gene PMA1.

FIG. 3A. Graphs illustrating relative H3K4me1 levels using ZipChIP that was performed on BY4741 set1Δ, wild-type (WT), using antibodies specific for H3K4me1 and histone H3 where PrimeTime qPCR probes were targeted toward the promoter (P), 5′-ORF (5), and 3′-ORF (3) of PYK1, MDH2, PMA1, and HMG1.

FIG. 3B. Graphs illustrating relative H3K4me1 levels using ZipChIP that was performed on BY4741 set1Δ, rad6Δ strain, using antibodies specific for H3K4me1 and histone H3 where PrimeTime qPCR probes were targeted toward the promoter (P), 5′-ORF (5), and 3′-ORF (3) of PYK1, MDH2, PMA1, and HMG1.

FIG. 3C. Graphs illustrating relative H3K4me1 levels using ZipChIP that was performed on BY4741 set1Δ, bre1Δ strain, using antibodies specific for H3K4me1 and histone H3 where PrimeTime qPCR probes were targeted toward the promoter (P), 5′-ORF (5), and 3′-ORF (3) of PYK1, MDH2, PMA1, and HMG1.

FIG. 4A. Schematic diagram of the four different regions analyzed for H3K4me3 and PKL-cMYC using region specific primers across the euchromatic gene ACT7 and the heterochromatic gene MULE.

FIG. 4B. Graphs illustrating relative H3K4me3 levels of the four different regions using region specific primers across the euchromatic gene ACT7 and the heterochromatic gene MULE, using ZipChIP. The H3K4me3 signal is normalized to input and histone H3.

FIG. 4C. Graphs illustrating relative PKL-cMYC levels of the four different regions using region specific primers across the euchromatic gene ACT7 and the heterochromatic gene MULE, using ZipChIP. ZipChIP analysis of PKL-cMYC was performed using a MYC specific antibody, and the signal was normalized to input. Three biological replicates were used for ZipChIP analysis. The error bars represent the standard error of the mean.

FIG. 5A. Blot showing the sonication of LNCaP chromatin resulting in a 100-200 base pair fragments and Histone H3 and H3K4 trimethylation levels are observed above background at 3-hydroxy-3-methyl-glutaryl-CoA reductase gene (HMGCR exon 1). 10 μL and 5 μL of isolated DNA from the LNCaP soluble lysate were run on a 1% agarose gel for 20 minutes at 100V.

FIG. 5B. Graph illustrating relative levels of Histone H3 at HMGCR exon 1 using ChIP analysis of two biological repeats. The levels of histone H3 were set relative to no antibody control (No Ab).

FIG. 5C. Graph illustrating relative levels of H3K4 trimethylation at HMGCR exon 1 using ChIP analysis of two biological repeats. The levels H3K4 trimethylation were set relative to no antibody control (No Ab).

FIG. 6A. Graphs illustrating relative levels of H3K4 trimethylation using both ZipChIP and standard ChIP where PrimeTime qPCR probes were targeted toward the promoter (P), 5′-ORF (5), and 3′-ORF (3) of an actively transcribed gene PYK1. ChIP analysis was performed on BY4741 wild-type (WT) strain using antibodies specific for H3K4me3 and histone H3. Input and histone H3 were used for normalization. ChIP analysis of H3K4 trimethylation in a set1Δ strain is relative to the H3K4me3 signal in a set1Δ strain for the promoter of each individual gene.

FIG. 6B. Graphs illustrating relative levels of H3K4 trimethylation using both ZipChIP and standard ChIP where PrimeTime qPCR probes were targeted toward the promoter (P), 5′-ORF (5), and 3′-ORF (3) of an actively transcribed gene PMA1. ChIP analysis was performed on BY4741 wild-type (WT) strain using antibodies specific for H3K4me3 and histone H3. Input and histone H3 were used for normalization. ChIP analysis of H3K4 trimethylation in a set1Δ strain is relative to the H3K4me3 signal in a set1Δ strain for the promoter of each individual gene.

FIG. 6C. Graphs illustrating relative levels of H3K4 trimethylation using both ZipChIP and standard ChIP where PrimeTime qPCR probes were targeted toward the promoter (P), 5′-ORF (5), and 3′-ORF (3) of a sub-telomere gene YFR057W. ChIP analysis was performed on BY4741 wild-type (WT) strain using antibodies specific for H3K4me3 and histone H3. Input and histone H3 were used for normalization. ChIP analysis of H3K4 trimethylation in a set1Δ strain is relative to the H3K4me3 signal in a set1Δ strain for the promoter of each individual gene.

FIG. 6D. Graphs illustrating relative levels of H3K4 trimethylation using both ZipChIP and standard ChIP where PrimeTime qPCR probes were targeted toward the promoter (P), 5′-ORF (5), and 3′-ORF (3) of an actively transcribed gene PYK1. ChIP analysis of H3K4 trimethylation in a WT strain is relative to the H3K4me3 signal in a WT strain for the promoter of each individual gene.

FIG. 6E. Graphs illustrating relative levels of H3K4 trimethylation using both ZipChIP and standard ChIP where PrimeTime qPCR probes were targeted toward the promoter (P), 5′-ORF (5), and 3′-ORF (3) of an actively transcribed gene PMA1. ChIP analysis of H3K4 trimethylation in a WT strain is relative to the H3K4me3 signal in a WT strain for the promoter of each individual gene.

FIG. 6F. Graphs illustrating relative levels of H3K4 trimethylation using both ZipChIP and standard ChIP where PrimeTime qPCR probes were targeted toward the promoter (P), 5′-ORF (5), and 3′-ORF (3) of a sub-telomere gene YFR057W. ChIP analysis of H3K4 trimethylation in a WT strain is relative to the H3K4me3 signal in a WT strain for the promoter of each individual gene.

FIG. 6G. Graphs illustrating relative levels of H3K4 trimethylation using both ZipChIP and standard ChIP where PrimeTime qPCR probes were targeted toward the promoter (P), 5′-ORF (5), and 3′-ORF (3) of an actively transcribed gene PYK1. H3K4me3 ChIP analysis in a WT strain is relative to the H3K4me3 signal for the promoter of YFR057W in a WT strain.

FIG. 6H. Graphs illustrating relative levels of H3K4 trimethylation using both ZipChIP and standard ChIP where PrimeTime qPCR probes were targeted toward the promoter (P), 5′-ORF (5), and 3′-ORF (3) of an actively transcribed gene PMA1. H3K4me3 ChIP analysis in a WT strain is relative to the H3K4me3 signal for the promoter of YFR057W in a WT strain.

FIG. 7A. Graph illustrating relative levels of JHD2-3×FLAG using ZipChIP where PrimeTime qPCR probes were targeted toward the ARS504, an origin of replication on chromosome V.

FIG. 7B. Graphs illustrating relative JHD2-3×FLAG where PrimeTime qPCR probes were targeted toward the ARS504. The fold difference of Jhd2-3×FLAG binding was calculated to compare the levels of Jhd2 at PYK1 and PMA1 to Jhd2 levels observed at ARS504.

FIG. 7C. Graph illustrating relative levels of RPH1-3×FLAG using ZipChIP where PrimeTime qPCR probes were targeted toward the ARS504, an origin of replication on chromosome V.

FIG. 8A. Graph showing Sir2 enrichment over five regions of the sub-telomere gene YFR057W using ZipChIP analysis. Region 1 was upstream of YFR057W in an intergenic region. Region 2 was in the YFR057W promoter. Region 3 and 4 were in the 5′ and 3′-ORF region of YFR057W, respectively. Region 5 was in the 3′-UTR of YFR057W.

FIG. 8B. Graph showing the localization at the 3′-ORF of two actively transcribed genes PYK1 (left) and PMA1 (right). Sir2 signal was set relative to a mock ZipChIP experiment with no antibody conjugated to the magnetic beads (No Ab).

FIG. 9A. Graphs illustrating two distinct patterns of H3K4 monomethylation using ZipChIP and global H3K4 methylation levels affected in RAD6 and BRE1 deletion strains where PrimeTime qPCR probes were targeted toward the promoter (P), 5′-ORF (5), and 3′-ORF (3) of ERG11 and HMG2.

FIG. 9B. Immunoblot showing global H3K4 monomethylation in rad6Δ and bre1Δ strains. Immunoblots of whole cell extracts from set1Δ, WT, rad6Δ, and bre1Δ strains were analyzed using methylation specific antibodies targeting H3K4 mono-, di- and trimethylation. A set1Δ strain was used as a negative control. H3 antibody was used as a loading control.

### BRIEF DESCRIPTION OF SEQUENCE LISTINGS

SEQ ID NO: 1: Probe for HMGCR exon1

SEQ ID NO: 2: Primer 1 for HMGCR exon1

SEQ ID NO: 3: Primer 2 for HMGCR exon1

SEQ ID NO: 4: Probe for PYK1 promoter

SEQ ID NO: 5: Primer 1 for PYK1 promoter

SEQ ID NO: 6: Primer 2 for PYK1 promoter

SEQ ID NO: 7: Probe for PYK1 5′

SEQ ID NO: 8: Primer 1 for PYK1 5′

SEQ ID NO: 9: Primer 2 for PYK1 5′

SEQ ID NO: 10: Probe for PYK1 3′

SEQ ID NO: 11: Primer 1 for PYK1 3′

SEQ ID NO: 12: Primer 2 for PYK1 3′

SEQ ID NO: 13: Probe for PMA1 promoter

SEQ ID NO: 14: Primer 1 for PMA1 promoter

SEQ ID NO: 15: Primer 2 for PMA1 promoter

SEQ ID NO: 16: Probe for PMA1 5′

SEQ ID NO: 17: Primer 1 for PMA1 5′

SEQ ID NO: 18: Primer 2 for PMA1 5′

SEQ ID NO: 19: Probe for PMA1 3′

SEQ ID NO: 20: Primer 1 for PMA1 3′

SEQ ID NO: 21: Primer 2 for PMA1 3′

SEQ ID NO: 22: Probe for MDH2 promoter

SEQ ID NO: 23: Primer 1 for MDH2 promoter

SEQ ID NO: 24: Primer 2 for MDH2 promoter

SEQ ID NO: 25: Probe for MDH2 5′

SEQ ID NO: 26: Primer 1 for MDH2 5′

SEQ ID NO: 27: Primer 2 for MDH2 5′

SEQ ID NO: 28: Probe for MDH2 3′

SEQ ID NO: 29: Primer 1 for MDH2 3′

SEQ ID NO: 30: Primer 2 for MDH2 3′

SEQ ID NO: 31: Probe for HMG1 promoter

SEQ ID NO: 32: Primer 1 for HMG1 promoter

SEQ ID NO: 33: Primer 2 for HMG1 promoter

SEQ ID NO: 34: Probe for HMG1 5′

SEQ ID NO: 35: Primer 1 for HMG1 5′

SEQ ID NO: 36: Primer 2 for HMG1 5′

SEQ ID NO: 37: Probe for HMG1 3′

SEQ ID NO: 38: Primer 1 for HMG1 3′

SEQ ID NO: 39: Primer 2 for HMG1 3′

SEQ ID NO: 40: Probe for ERG11 promoter

SEQ ID NO: 41: Primer 1 for ERG11 promoter

SEQ ID NO: 42: Primer 2 for ERG11 promoter

SEQ ID NO: 43: Probe for ERG11 5′

SEQ ID NO: 44: Primer 1 for ERG11 5′

SEQ ID NO: 45: Primer 2 for ERG11 5′

SEQ ID NO: 46: Probe for ERG11 3′

SEQ ID NO: 47: Primer 1 for ERG11 3′

SEQ ID NO: 48: Primer 2 for ERG11 3′

SEQ ID NO: 49: Probe for HMG2 promoter

SEQ ID NO: 50: Primer 1 for HMG2 promoter

SEQ ID NO: 51: Primer 2 for HMG2 promoter

SEQ ID NO: 52: Probe for HMG2 5′

SEQ ID NO: 53: Primer 1 for HMG2 5′

SEQ ID NO: 54: Primer 2 for HMG2 5′

SEQ ID NO: 55: Probe for HMG2 3′

SEQ ID NO: 56: Primer 1 for HMG2 3′

SEQ ID NO: 57: Primer 2 for HMG2 3′

SEQ ID NO: 58: Probe for YFR057W upstream intergenic

SEQ ID NO: 59: Primer 1 for YFR057W upstream intergenic

SEQ ID NO: 60: Primer 2 for YFR057W upstream intergenic

SEQ ID NO: 61: Probe for YFR057W promoter

SEQ ID NO: 62: Primer 1 for YFR057W promoter

SEQ ID NO: 63: Primer 2 for YFR057W promoter

SEQ ID NO: 64: Probe for YFR057W 5′

SEQ ID NO: 65: Primer 1 for YFR057W 5′

SEQ ID NO: 66: Primer 2 for YFR057W 5′

SEQ ID NO: 67: Probe for YFR057W 3′

SEQ ID NO: 68: Primer 1 for YFR057W 3′

SEQ ID NO: 69: Primer 2 for YFR057W 3′

SEQ ID NO: 70: Probe for YFR057W 3′-UTR

SEQ ID NO: 71: Primer 1 for YFR057W 3′-UTR

SEQ ID NO: 72: Primer 2 for YFR057W 3′-UTR

SEQ ID NO: 73: Probe for ARS504

SEQ ID NO: 74: Primer 1 for ARS504

SEQ ID NO: 75: Primer 2 for ARS504

SEQ ID NO: 76: Forward primer for ACTT; region 1

SEQ ID NO: 77: Reverse primer for ACTT; region 1

SEQ ID NO: 78: Forward primer for ACTT; region 2

SEQ ID NO: 79: Reverse primer for ACTT; region 2

SEQ ID NO: 80: Forward primer for ACTT; region 3

SEQ ID NO: 81: Reverse primer for ACTT; region 3

SEQ ID NO: 82: Forward primer for ACTT; region 4

SEQ ID NO: 83: Reverse primer for ACTT; region 4

SEQ ID NO: 84: Forward primer for MULE; region 1

SEQ ID NO: 85: Reverse primer for MULE; region 1

SEQ ID NO: 86: Forward primer for MULE; region 2

SEQ ID NO: 87: Reverse primer for MULE; region 2

SEQ ID NO: 88: Forward primer for MULE; region 3

SEQ ID NO: 89: Reverse primer for MULE; region 3

SEQ ID NO: 90: Forward primer for MULE; region 4

SEQ ID NO: 91: Reverse primer for MULE; region 4

### DETAILED DESCRIPTION

For the purposes of promoting an understanding of the principles of the present disclosure, reference will now be made to the embodiments illustrated in the drawings, and specific language will be used to describe the same. It will nevertheless be understood that no limitation of the scope of this disclosure is thereby intended.

This application claims the benefit of U.S. Provisional Patent Application No. 62/090,087 filed on Dec. 10, 2014 the disclosure of which is incorporated herein by reference in its entirety. Relative to the provisional application, the name of the claimed ChIP invention has been changed; originally referred to as “iChIP” in U.S. Provisional Patent Application No. 62/090,087 it is now referred to as “ZipChIP.”

The disclosure provides details on a new method and novel buffers for purifying and analyzing a sample from a cell. In certain aspects, the sample is chromatin. The method overcomes various deficiencies in the art, and allows for fast extraction and analysis of DNA within the chromatin region of interest, by allowing for direct analysis on a bead or support. In certain aspects the chromatin comprises DNA and histones. In certain aspects the chromatin is a region of interest because of a post translational modification event. In other aspects the chromatin region is of interest because of the presence of a biomolecule. In certain aspects the chromatin region is of interest because it is associated with a disease state. The chromatin is extracted using the novel buffers and reagents and captured on a bead or support. In other aspects the molecule of interest is RNA. Additionally, the sample may comprise nucleic acids and additional proteins including transcription factors, replication promoting proteins, coactivators, etc. The composition of the buffers and reagents allows for direct analysis of the captured nucleic acids immediately without any additional steps. In certain instances the captured chromatin may be heated to elute it from the bead or support prior to performing an analysis.

By “chromatin” refers to a complex of molecules comprising DNA, RNA, and proteins. More specifically chromatin refers to a protein-DNA complex that packages DNA in the nucleus of cells. The basic unit of chromatin is the nucleosome, which is composed of 146 base pairs of DNA wrapped around an octamer of histone proteins, and other biomolecules may be associated with this complex.

By “chromatin immunoprecipitation or ChIP” refers to an experimental technique to understand the relationship between nucleic acids and proteins in the nucleus, wherein the nucleic acids and proteins are cross-linked, the nucleic acids are sheared, the proteins are captured by antibodies conjugated to a support, the nucleic acids are uncross-linked from the protein

By “polymerase chain reaction or PCR” refers to a molecular biology method wherein a single copy of DNA or RNA is amplified or copied several times generating upwards of millions of copies to allow for sequencing of the DNA.

By “eukaryotic cell” refers to a cell having a nucleus and other organelles enclosed in a membrane. Non-limiting examples of eukaryotic cells are cells found in plants, fish, zebra fish, mice, humans, yeast, dogs, cows, etc.

By “prokaryotic cell” refers to a cell that lacks a membrane bound nucleus. A non-limiting example of a prokaryotic cell is a bacterium.

By “posttranslational modification” refers to any modification of a protein after translation. Some examples of posttranslational modifications are folding, cutting, methylation, acetylation, myristoylation, palmitoylation, isoprenylation or prenylation, farnesylation, geranylgeranylation, glypiation, lipoylation, attaching a flavin, attaching a heme C, phosphopantetheinylation, phosphorylation, retinylidene Schiff base formation, acylation, alkylation, amide bond formation, amidation, arginylation, polyglutamylation, polyglycylation, butyrylation, gama-carboxylation, glycosylation, malonylation, hydroxylation, iodination, nucleotide addition, oxidation, phosphoramidate formation, adenylylation, propionylation, pyroglutamate formation, S-glutathionylation, S-nitrosylation, succinylation, sulfation, glycation, biotinylation, pegylation, ISGylation, SUMOylation, ubiquitination, Neddylation, Pupylation, citrullination, deadmidation, eliminylation, carbamylation, disulfide bridges, proteolytic cleavage, and racemization or proline, serine, alanaine, or methionine.

By “protein” refers to a molecule comprising amino acids. The proteins described herein may be naturally occurring as a biomolecule, non-naturally occurring biomolecule, or synthetic.

By “biomolecule” refers to any molecule produce by a living organism. Non-limiting examples are DNA, RNA, mRNA, protein, antibodies, viruses, bacteria, cholesterol, sterol, transcription factors, remodelers, complexes that are a combination of these identified elements, etc. It should also be noted that most, if not all, biomolecules may be made synthetically or at the very least deliberately in laboratory assays.

By “antibody” refers to a type of protein which has a distinct shape and domains known in science. Antibodies are produced in vivo naturally or deliberately in the lab (and may be made in a test tube environment) and used to identify and bind to foreign objects such as a virus or bacteria. Antibodies may be deliberately generated in laboratory assays to bind to a specific biomolecule or molecule of interest. In a practical application setting, antibodies may be used as therapeutics or as capture agents to purify or isolate a protein of interest within a mixture.

By “chromatin-antibody-bead complex” refers to a complex generated with chromatin is bound to an antibody, and the antibody is bound to a bead.

The novel method includes a method of analyzing chromatin comprising; providing solubilized chromatin and a plurality of beads having at least one antibody bound to individual beads; conjugating the solubilized chromatin to the at least one antibody and generating a chromatin-antibody-bead complex; immunoprecipitating the chromatin-bead complex; washing the chromatin-antibody-bead complex with SB-140 wash buffer, SB-500 wash buffer, and 1×PBS at least once with each; resuspending the chromatin-antibody-bead complex in water; and performing polymerase chain reaction on the chromatin-antibody-bead complex.

In certain aspects the plurality of beads may all be the same size and material of bead or there may be a heterogeneous mixture of beads varying in size or varying in material. In certain aspects the bead is a magnetic bead. In other aspects the bead may comprise agarose, sepharose, or a metal. The bead may range in a size between about 50 nanometers (nm) to about 1 millimeter (mm). The bead may be conjugated to at least one antibody specific to a type or family of protein. The bead may be conjugated with a plurality of antibodies specific to a type or family of protein. The bead may be conjugated to a plurality of antibodies, wherein the antibodies are a heterogeneous mixture where the antibodies detect at least two different protein types or protein families. The antibodies are distributed over the bead. If more than one type of antibody is present on a single bead, the ratio of the antibodies may be 1:1, 2:1, 3:1, 4:1, 5:1, 6:1, 7:1, 8:1, 9:1, 10:1, and so. The antibodies may be distributed on the bead evenly, randomly, or separated geographically along the axes of the bead.

The antibody or antibody fragment associated with the bead will be specific to the analysis being performed. One of skill will recognize this, and would not require undo experimentation to substitute one antibody for another. The antibody conjugated to the bead may be directly bound or bound indirectly through a linker. The linker may be a peptide, protein, chemical bond, small molecule, amino acids, or nucleic acids. In one aspect the linker is a protein A. In other aspects the linker may be a peptide. Should a plurality of antibodies be bound to the bead, a first primary antibody or primary antibody fragment may be used to detect a first protein of interest. A second primary antibody or primary antibody fragment thereof may be used to detect a second protein of interest. A third primary antibody or primary fragment thereof may be used to detect a third protein of interest, and so. In another aspect, the second antibody may be used to bind a reporter to signal that a certain protein has bound to the bead before any further steps occur. A reporter may be a fluorescent molecule, a non-fluorescent molecule such as a Raman tag, or another bead. In certain aspects the system may be designed so that one bead comprises one antibody, wherein bead type one may be bound directly or indirectly to a first antibody and a second bead type may be bound directly or indirectly to a second antibody and so on. The difference in bead type may be size, material or a combination thereof.

In other aspects, the bead may be bound to another binding molecule that is not an antibody. This binding molecule is used to capture and purify its target. This binding molecule may be a biomolecule, a peptide, a protein or fragment thereof, a ligand or fragment thereof, a receptor or fragment thereof, a protein that indiscriminately captures proteins due to a “sticky” characteristic, a biotin-streptavidin interaction, protein comprising at least one chromatin binding domains, histone binding protein, protein that recognizes at least one posttranslational modification or fragment thereof, a transcription factor or fragment thereof, coactivator of fragment thereof, DNA replication factor or fragment thereof, nucleic acids of a predetermined sequence, siRNA, mRNA, or a combination thereof. The binding molecule may be directly or indirectly bound through a linker to the bead.

In certain embodiments, the binding molecule such as an antibody or fragment thereof, nucleic acids, ligands, proteins, etc may be bound to a support other than a bead. This support may comprise a magnetic material, sepharose, agarose, plastic, or some other metal. The support is immersed into the sample suspension and the fluid is moved over the support to induce an interaction. The bound sample may remain bound to the support through the binding molecule, and analysis may be performed directly on the sample bound to the support. The binding molecule may be bound directly or indirectly through a linker to the support.

An analysis of the bound sample, which may be a chromatin complex, may be performed by PCR, quantitative real-time PCR, or reporter. The analysis may be performed solely on DNA or RNA bound to the bead or support. The chromatin, RNA, or DNA may be heated and eluted to remove it from the bead or support before analysis.

The buffers and reagents used may be any known in the art, but the best mode is to use the buffers and reagents identified and detailed below.

The method may be used to identify chromatin, chromatin associated proteins, or proteins bound to DNA or RNA having at least one posttranslational modification. For instance, the method may be used to identify a histone having a posttranslational modification that would be associated with up regulating or down regulating transcription and translation in that region. In other instances, the method may be used to identify chromatin, chromatin associated proteins, or proteins that do not have at least one posttranslational modification. For instance, the method may be used to identify a protein such as a transcription factor, co-factor, co-activator, or remodeler associated with a specific stretch of DNA. In one aspect, the assay may be used to identify a segment of DNA that is associated with cancer. In one aspect, the assay may be used to verify that a gene was delivered and incorporated into a segment of DNA successfully. In another aspect, the method may be used to identify the insertion of DNA from a virus. By selecting the appropriate antibody or binding molecule, the method may also be used to detect modified DNA, for example methylated or hydroxymethylated DNA. In other aspects, the method may be used to identify histone modifications or posttranslational modifications, identify histone proteins, identify modified DNA, identify transcription factors, identify coactivators, identify RNA polymerase, identify chromatin associated proteins, or a combination thereof. In another aspect, the method and system may be used to immunoprecipitate RNA and proteins associated with RNA such as RNA polymerase.

One exemplary embodiment of the procedure for performing the method on yeast cells is as follows: 1) Grow culture(s) of yeast. For the S. cerevisiae ChIP analysis in this description 100 mL culture size was used; however, this can be adjusted depending on what is targeted for ChIP and number of targets for ChIP 2) Crosslink cells with a cross-linking agent such as formaldehyde. Incubate at room temperature, mixing occasionally 3) Collect cells with centrifugation. Wash at least once H₂O. Collect cells again with centrifugation. Transfer culture to a microcentrifuge tube with H₂O, centrifuge, flash freeze cell pellet with liquid nitrogen, and store at −80° C. To ensure sufficient cell lysis, the cell pellets should be no more than 50% of starting culture. If necessary, one could store cell pellet at −80° C. till ready for next steps. The Preparation of soluble chromatin may be performed using the following steps resuspend a portion of cells in SB-140 lysis buffer, add an object such as a glass beads to the top of the solution. Beat beads in cold block at least once with rest between additional cycles, next remove liquid from beads from the lysate. One method for removing the beads is to create a hole in bottom of tube to separate liquid from beads by placing tube into an additional microcentrifuge tube and centrifuging for a time at top speed. The cell lysate may be sonicated, for example by using a Misonix sonicator 3000: 30% output, 90% duty cycle, 10 seconds, 6 times with 10 second rest), and place samples on dry ice or another cold source immediately after sonication. The optionally sonicated lysate may be separated in at least two fractions by use of a centrifuge, for examples samples may be centrifuged for a time at top speed and at 4° C. The resulting supernatant may be transferred to a new tube, and then centrifuged at top speed for a time at a temperature of 4° C. The resulting supernatant may be transferred to a new tube. The resulting sample is a whole cell extract (WCE) which includes soluble chromatin. If the resulting lysate is not clear after the centrifugation, the sample can be centrifuged at least one more time for a number of minutes at top speed at 4° C. If it is convenient to do so the soluble chromatin may be stored at −20° C. The next step in the exemplary process is to transfer (about 6.25%) of WCE to a microcentrifuge tube, and if necessary to purify DNA for input using a kit for example a QIAGEN PCR purification kit (28106).

### Immunoprecipitation

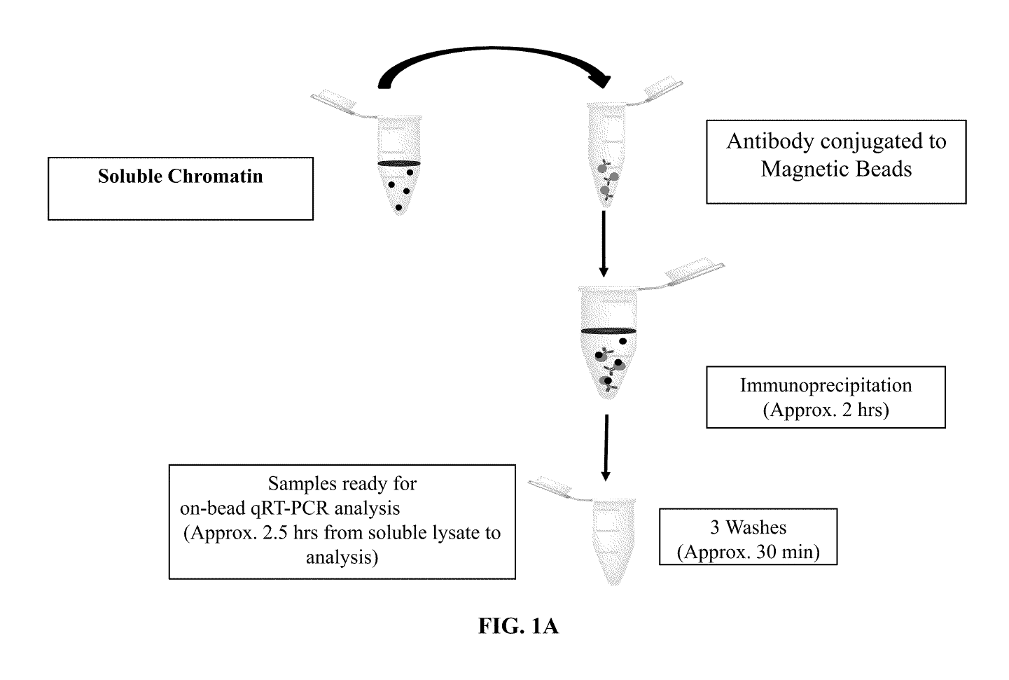

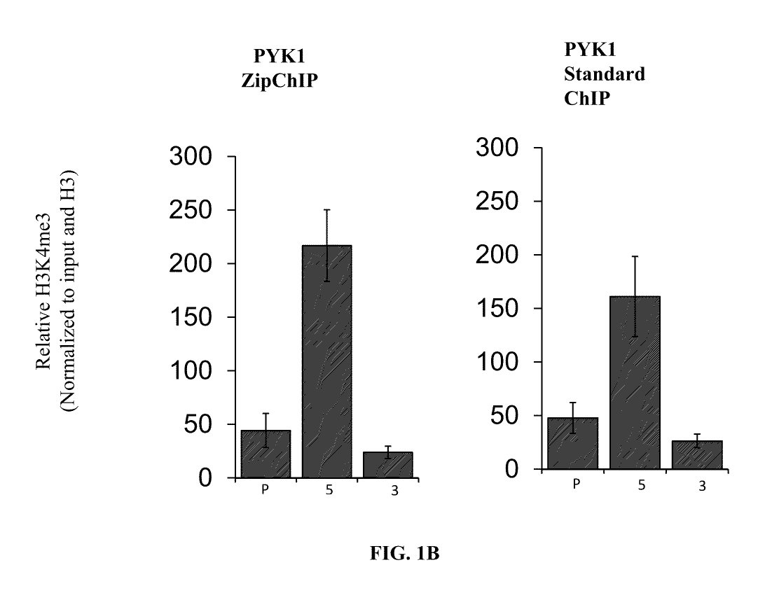

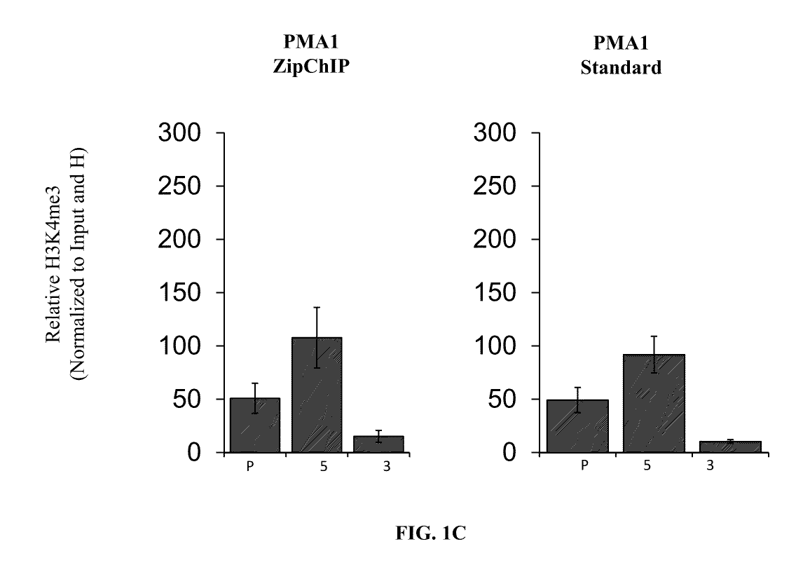

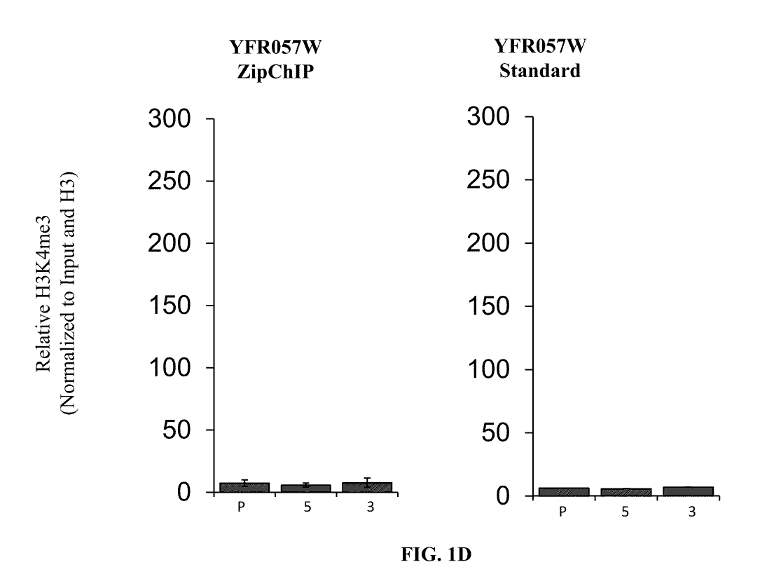

An exemplary process for conjugating elements in the sample to for example magnetic beads, is to add a desirable amounts of Protein G magnetic beads (Dynabeads Life Technology 10004D), 1×PBS(-K), and antibody in a microcentrifuge tube. The mixture may be incubated with the antibody and beads at room temperature with rotation for a time. Referring now for example to FIGS. 1, 4, 6, and 9, for the α-H3K4me3, α-H3K4me1, α-H3, and α-FLAG antibodies used and 1 μL antibody serum can be used. It will be appreciated that the amount of antibody and antibody serum may need to be optimized for each protein targeted for ChIP. Such optimization may be readily determined by the skilled artisan. The next step in the exemplary process is to separate the beads from 1×PBS(-K) buffer. If magnetic beads are used the beads may be separated by placing the microcentrifuge tube on a magnetic separator. Allow beads to adhere to side of tube and remove the supernatant without disrupting the magnetic beads. As needed 1×PBS(-K) may be added to the whole cell extract lysate to the magnetic beads conjugated to the antibody. It will be appreciated that the volume of lysate used for immunoprecipitation may need to be optimized for each protein target. Such optimization may be readily determined by the skilled artisan. In some embodiment in order to reduce background, the lysate may be precleared with 5 μL magnetic beads not conjugated to antibody by rotating lysate and beads for a time at 4° C., Next the lysate and antibody-conjugated beads are rotated for between 30 minutes and 4 hours, but may be longer at 4° C. After immunoprecipitation, place samples on magnetic separator and remove supernatant. As a wash buffer cold SB-140 may be added, and the mixture rotated for a number minutes at room temperature. Samples may be placed on a magnetic separator to aid in the removal of the supernatant. Cold SB-500 wash buffer may be added in a second wash step and the mixture rotated for a number of minutes at room temperature. The samples may then be placed on magnetic separator, followed by removal of the supernatant. A third wash may be run by adding 1×PBS(-K), followed by rotating the mixture for a number of minutes at room temperature. The samples may be placed on a on magnetic separator to remove the supernatant. To reduce background noise in the analytical step one may optionally use the 1×PBS(-K) in the third wash to transfer magnetic beads to a new microcentrifuge tube. The beads may then be resuspended in cleans solution, for example ddH₂O.

In some embodiments the processed is sample is subjected to qRT-PCR Analysis. In some embodiments qRT-PCR analysis may be carried out using a commercial kit such as TAQMAN MASTER MIX (Life Technologies 4369510) (for example: 5 μL TaqMan Master Mix, 3.5 μL H₂O, 0.5 μL primer/probe mix (IDT), and DNA, either immunoprecipitation on magnetic beads or purified DNA for input). In some embodiments the magnetic bead mixtures may be vortexed immediately prior to aliquoting to ensure consistent suspension.

The following are exemplary embodiments of the reagents and their ingredients used in the exemplary procedures: SB-140 Lysis Buffer: 50 mM HEPES-KOH, pH 7.5; 140 mM NaCl; 1 mM EDTA; 0.1% Triton X-100; and 0.1% Na Deoxycholate. SB-140 Wash Buffer: 50 mM HEPES-KOH, pH 7.5; 140 mM NaCl; 1 mM EDTA; and 0.1% Triton X-100. SB-500 Wash Buffer: 50 mM HEPES-KOH, pH 7.5, 500 mM NaCl; 1 mM EDTA; and 0.1% Triton X-100. 10×PBS(-K) Stock, pH 7.0: 390 mM NaH₂PO₄—H₂O; 323 mM Na₂HPO₄.7H₂O; and 1.54 M NaCl.

While the above reagents are exemplary and best mode for the above method, it is understood that other detergents may be used as well. A non-limiting list of detergents includes nonyl phenoxypolyethoxylethanol (NP-40), tween 20, SDS, TritonX-114, Brij-35, Brij-58, Tween 80, octyl glucoside, octyl thioglucoside, CHAPS, CHAPSO, n-Dodecyl-beta-D-Maltoside, Sodium Cholate, Sodium deoxygcholate, any amphiphatic molecule with characteristics of a detergent, or a combination thereof.

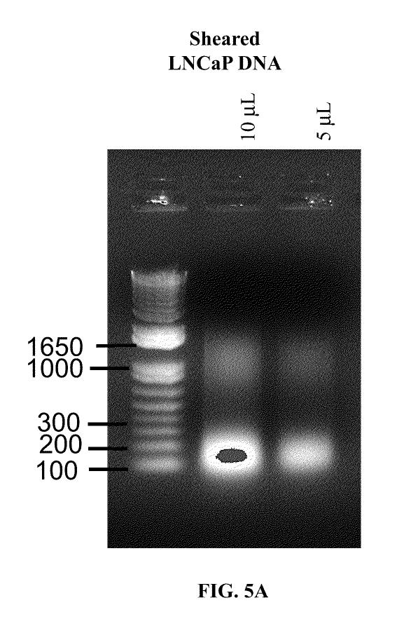

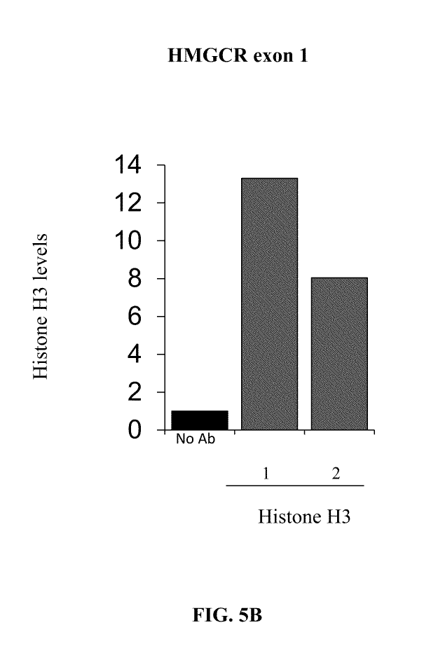

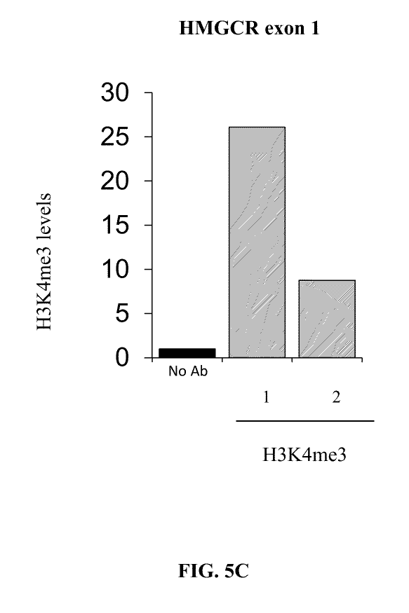

Another exemplary protocol using the ZipChIP novel buffers and methods using mammalian cells is as follows and demonstrated in FIG. 5. This protocol is not meant to be limiting in any way, and those of skill in the art will recognize that known optimizations and substitutions may be made that would be obvious.

### Cell Culture Reagents and Formaldehyde Cross-Linking

The epithelial prostate cancer cell line LNCaP was purchased from the American Tissue Culture Collection (ATCC: Manassas, Va.). The cells were maintained at 37° C. with 5% carbon dioxide. The cells were maintained in RPMI-1640 medium supplemented with 10% fetal bovine serum (FBS), 1% HEPES buffer, 1% sodium pyruvate, and 1% non-essential amino acids (NEAA). 20×10⁷ cells were harvested using a 0.25% trypsin-EDTA solution. The cells were centrifuged at 150×g for 7 minutes and the supernatant was removed. The cell pellet was resuspended in 37 mL of a 1% formaldehyde solution diluted in PBS and fixed for 8 minutes with gentle rocking at room temperature. The cells were then centrifuged at 300×g for 7 minutes and washed with 50 mL ice-cold PBS twice.

### Generating Soluble Chromatin for LNCaP Cells

The cells were responded in 10 mL ice cold 1×PBS. They were resuspended by pipetting up and down and then pelleted at 1200 rpm for 5 minutes at 4° C. The cells were then washed with 3 mL ice-cold Wash Buffer I (0.25% Triton X-100; 10 mM EDTA, pH 8; 0.5 mM EGTA, pH 7.5; 10 mM HEPES, pH 7.5) with protease inhibitors (0.2 μg leupeptin, aprotinin, and pepstatin A, 0.2 mM PMSF) and centrifuged at 2100 rpm for 5 minutes at 4° C. The pelleted cells were washed with 3 mL ice-cold Wash Buffer II (0.2 M NaCl; 1 mM EDTA, pH 8; 0.5 mM EGTA, pH 7.5; 10 mM HEPES, pH 7.5) with protease inhibitors and centrifuged at 2100 rpm for 5 minutes at 4° C. The cells were then resuspended in 1 mL Lysis Buffer I (150 mM NaCl; 25 mM Tris, pH 7.5; 5 mM EDTA; 1% Triton X-100; 0.1% SDS; 0.5% sodium deoxycholate) with protease inhibitors. The resuspended cells were split into two 500 mL fractions. Each fraction were sonicated three times each (Misonix sonicator: Power 6, 10 seconds, 6 times with 10 second rest) to shear the chromatin. After each sonication the cells were placed directly on dry ice for 10 seconds. After sonication, the samples were centrifuged at 14000 rpm for 10 minutes at 4° C. The supernatant for each of the two samples were then combined and mixed.

### Exemplary LNCaP ZipChIP Protocol

Before immunoprecipitation, two 225 μL of soluble lysate samples were incubated with 5 μL magnetic beads (Dynabeads, Life Technologies 10004D) for 30 minutes with rotation at 4° C. to preclear the lysate. After the lysate samples were precleared, the samples were combined and mixed. During this time 1 μL histone H3 antibody (Abcam; ab1791) and 1 μL H3K4 trimethylation specific antibody (Millipore; 07-473) was individually incubated with 10 μL magnetic beads (Dynabeads, Life Technologies 10004D) for 30 minutes with rotation at room temperature in 200 μL 1×PBS(-K), pH 7.0 (39 mM NaH₂PO₄.H₂O; 32.3 mM Na₂HPO₄.7H₂O; 150 mM NaCl) to conjugate the antibody to the beads. 200 μL 1×PBS(-K) was also incubated with 10 μL magnetic beads as a no antibody control. The antibody bound beads and no antibody control were then each rotated with 100 μL soluble lysate and 100 μL 1×PBS(-K). The beads were incubated with the soluble lysate for 2 hours at 4° C. with rotation. The beads were then washed at room temperature for 5 minutes each with sFA-140 wash buffer (50 mM HEPES-KOH, pH 7.5; 140 mM NaCl; 1 mM EDTA; 0.1% Triton X-100), sFA-500 wash buffer (50 mM HEPES-KOH, pH 7.5; 500 mM NaCl; 1 mM EDTA; 0.1% Triton X-100), and finally with 1×PBS(-K). 50 μL water was used to resuspend the beads.

### Optional Testing of Sonication Efficiency

To prepare a DNA sample to test the sonication efficiency, 25 μL of soluble chromatin was diluted with 370 μL TE, pH 8.0 (10 mM Tris; 1 mM EDTA) and reversed cross-linked overnight at 65° C. After reverse cross-linking, the DNA was ethanol precipitated and dried. The dried DNA was resuspended in 180 μL TE, pH 8.0. RNase (20 μg) was then added and incubated for 30 minutes at 37° C. 20 μL Proteinase K buffer (0.1M Tris-HCl, pH 7.8; 0.05 M EDTA; 5% SDS) was added followed by the addition of 20 μg Proteinase K (Sigma P2308). This was then incubated for 1 hour at 42° C. The DNA was extracted with two phenol/chloroform/IAA washes. The DNA was again ethanol precipitated, dried, and resuspended in 50 μL water.

### Exemplary LNCaP Real-Time PCR and Statistical Analysis

qRT-PCR was performed using the StepOnePlus Real-Time PCR System (Life Technologies). To ensure equal amount of DNA loaded into the reaction mixture, the samples were regularly vortexed to ensure suspension of the beads. 1 μL of the on-bead mixture were used for each of the three technical repeats. The DNA was amplified using TaqMan Master Mix (Life Technologies 4369510) and PrimeTime probe sets (Integrated DNA Technologies), which are described in Table 0.

TABLE 0
Probe sets for ZipChIP analysis
in LNCaP cells

| Primer name | Sequence 5′-3′ |
|---|---|
| HMGCR exon 1 | /56-FAM/ATTGTGTGT/ZEN/GGGACCGTAATGGCT/ |
| | 3IABkFQ/ (SEQ ID NO: 1) |
| | AAATGCCCGGCAGCTTG (SEQ ID NO: 2) |
| | CTGCCAATGCTGCCATAAGT (SEQ ID NO: 3) |

### Examples of Material and Methods

Yeast Strains:

All yeast strains used in this study are described in Table 1. All strains were grown overnight to saturation in YPD media. They were then back diluted to OD₆₀₀ 0.1-0.2 in 100 mL YPD and grown at 30° C. to mid-log phase (OD₆₀₀ 0.6-0.8).

TABLE 1
Yeast strains used for ZipChIP and standard ChIP procedures

| Yeast Strain | Genotype | Reference |
|---|---|---|
| BY4741 | MATα his3Δ leu2Δ0 LYS2 met15Δ0 ura3Δ0 | Open Biosystems |
| SDBY1210 | BY4741: MATα his3Δ leu2Δ0 LYS2 met15Δ0 ura3Δ0 | (1) |
| | set1Δ::HygMX | |
| SDBY1107 | BY4741: MATα his3Δ leu2Δ0 LYS2 met15Δ0 ura3Δ0 JHD2 | (2) |
| | 3xFLAG::KanMX | |
| SDBY1319 | BY4741: MATα his3Δ leu2Δ0 LYS2 met15Δ0 ura3Δ0 RPH1 | This study |
| | 3xFLAG::KanMX | |
| rad6Δ | BY4741: MATα his3Δ leu2Δ0 LYS2 met15Δ0 ura3Δ0 | Open Biosystems |
| | rad6Δ::KanMX | |
| bre1Δ | BY4741: MATα his3Δ leu2Δ0 LYS2 met15Δ0 ura3Δ0 | Open Biosystems |
| | bre1Δ::KanMX | |

Formaldehyde Cross-Linking and Generating Soluble Chromatin from S. cerevisiae:

The generation of soluble chromatin was described previously. The cultures were cross-linked with 1% formaldehyde for 15 min at room temperature, mixing occasionally. The cells were then collected with centrifugation and washed. The cells were again collected with centrifugation, transferred to a microcentrifuge tube, and collected with centrifugation. The cell pellet was flash frozen in liquid nitrogen and stored at −80° C. The cell pellets were resuspended in 400 μL SB-140 lysis buffer (SB-140 refers to the FA-140 buffer used for Saccharomyces cerevisiae; 50 mM HEPES-KOH, pH 7.5; 140 mM NaCl; 1 mM EDTA; 0.1% Triton X-100, and 0.1% sodium deoxycholate). The cells were lysed by bead-beating with glass beads (BioSpec 11079105). The lysate was separated from the beads and sonicated (Misonix sonicator: Power 6, 10 seconds, 6 times with 10 second rest) to shear the chromatin. Directly after sonication, the lysate was placed on dry ice for 10 sec. The lysates were centrifuged for 5 min at 4° C. at top speed and then for 10 min at 4° C. at top speed to separate soluble and insoluble chromatin (˜400 μL). 25 μL (6.25%) of soluble chromatin was removed and processed for DNA input using a PCR purification kit (Qiagen 28106). The remaining soluble chromatin can be immediately used for the immunoprecipitation or it can be stored at −20° C.

S. cerevisiae ZipChIP:

Individual antibodies (1 μL H3, Abcam ab1791; 1 μL H3K4me3, Millipore 07-473; 1 μL H3K4me1, Active Motif 39297; 1 μL M2 FLAG, Sigma F, 1804; 10 μL Sir2 (y-80), Santa Cruz sc-25753) were conjugated to 10 μL Protein-G magnetic beads (Dynabeads, Life Technologies 10004D) for about 30 min. To determine the amount of antibody used per immunoprecipitation, different volumes of antibody were tested to determine the amount that would result in the best signal to noise ratio. The antibody bound beads were then rotated with soluble lysate (100 μL for H3; 25 μL for H3K4me3; 300 μL for H3K4me1; 200 μL for FLAG; 500 μL for Sir2; for H3 and H3K4me3 the soluble lysate was brought up to 200 μL total volume with 1×PBS(-K)) for 2 hr at 4° C. The beads were then washed at room temperature for 5 min each with sFA-140 wash buffer (50 mM HEPES-KOH, pH 7.5; 140 mM NaCl; 1 mM EDTA; 0.1% Triton X-100), sFA-500 wash buffer (50 mM HEPES-KOH, pH 7.5; 500 mM NaCl; 1 mM EDTA; 0.1% Triton X-100), and with 1×PBS(-K), pH 7.0 (39 mM NaH₂PO₄—H₂O; 32.3 mM Na₂HPO₄.7H₂O; 154 mM NaCl). 50 μL water was used to resuspend the beads.

S. cerevisiae Standard ChIP:

The standard ChIP protocol was performed as previously described with slight modifications. Soluble lysate (100 μL for H3; 25 μL for H3K4me3; 200 μL for FLAG; for H3 and H3K4me3 the soluble lysates were brought up to 200 μL total volume with sFA-140 lysis buffer) was rotated with 1 μL antibody (H3, Abcam ab1791; H3K4me3, Millipore 07-473; M2 FLAG, Sigma F, 1804) overnight at 4° C. with protease inhibitors (0.2 μg leupeptin, aprotinin, and pepstatin A, 0.2 mM PMSF). Similar to the antibodies, to determine the amount of lysate used per immunoprecipitation, different volumes of lysate were tested to determine the amount that would result in the best signal to noise ratio. 12 μL Sepharose Protein-G beads (GE Healthcare Life Sciences 17-0618-02) was added to each lysate and incubated for 1 hr at 4° C. The beads were then washed at room temperature for 10 min with sFA-140 wash buffer, sFA-500 wash buffer, and with LiCl/NP40 (10 mM Tris-HCl, pH 8.0; 250 mM LiCl; 0.5% NP-40; 0.5% sodium deoxycholate; 1 mM EDTA). The bound protein and DNA was eluted from the beads using 200 μL elution buffer (1% SDS; 0.1 M NaHCO₃) by briefly vortexing the beads followed by 15 min rotation at room temperature. This was repeated, and the two elution fractions were combined. After combining the two elutions, 16 μL 5 M NaCl was added to each sample. For DNA input, 10 μL soluble chromatin was used. Both the elutions and the inputs were reverse cross-linked overnight at 65° C. The DNA was then ethanol precipitated and dried. 180 μL TE, pH 8.0 (10 mM Tris; 1 mM EDTA) was used to resuspend the DNA followed by RNase treatment (20 μg; Sigma R6513) for 30 min at 37° C. 20 μL Proteinase K buffer (0.1 M Tris-HCl, pH 7.8; 0.05 M EDTA; 5% SDS) was added followed Proteinase K treatment (20 μg; Sigma P2308) for 1 hr at 42° C. The DNA was extracted with two phenol/chloroform/IAA washes. The DNA was again ethanol precipitated, dried, and resuspended in 50 μL water.

S. cerevisiae Real-Time PCR and Statistical Analysis:

qRT-PCR was performed using the StepOnePlus Real-Time PCR System (Life Technologies). To ensure equal amounts of DNA loaded, the samples were regularly vortexed to ensure suspension of the beads. Also to be noted, low levels (0.1% Triton X-100) must be used in the SB-140 wash buffer and SB-500 wash buffer to reduce background fluorescence caused by detergents when performing an on-bead PCR reaction. 1 μL of the on-bead mixture and standard ChIP DNA were used for each of the three technical repeats for each of the three biological repeats. The DNA was amplified using TaqMan Master Mix (Life Technologies 4369510) and PrimeTime probe sets (Integrated DNA Technologies) described in Table 2. The data were analyzed using the ΔΔCt method. The mean relative fold changes, s.e.m., and statistical significance values are found in Tables 4-13.

TABLE 2
Probe sets for ZipChIP and standard
ChIP analysis in S. cerevisiae

| Primer | |
|---|---|
| name | Sequence 5′-3′ |
| PYK1 | /56-FAM/CGAATATCG/ZEN/TTTTGATGGCGAGCCTTT/ |
| promoter | 3IABkFQ/ (SEQ ID NO: 4) |
| | AGAGGTTCTTGGAAATGAAAAGTTAC (SEQ ID NO: 5) |
| | CCTTCCCATATGATGCTAGGTAC (SEQ ID NO: 6) |
| PYK1 5′ | /56-FAM/AGAATTGTA/ZEN/CCCAGGTAGACCATTGGC/ |
| | 3IABkFQ/ (SEQ ID NO: 7) |
| | TTCTGATTTCTGGACCCTTGG (SEQ ID NO: 8) |
| | CATTGACAACGCCAGAAGTC (SEQ ID NO: 9) |
| PYK1 3′ | /56-FAM/CTCTCACTT/ZEN/GTACAGAGGTGTCTTGGGA/ |
| | 3IABkFQ/ (SEQ ID NO: 10) |
| | TCAGAGACAGGTTCCTTTTCG (SEQ ID NO: 11) |
| | TTACCAGATGCCCAAGAGC (SEQ ID NO: 12) |
| PMA1 | /56-FAM/CCGCTTATG/ZEN/CTCCCCTCCATTAGTT/ |
| promoter | 3IABkFQ/ (SEQ ID NO: 13) |
| | TGACGAAACGTGGTCGATG (SEQ ID NO: 14) |
| | AAATTAGATGTTAGACGATAATGATAGGAC |
| | (SEQ ID NO: 15) |
| PMA1 5′ | /56-FAM/TTGCCGCCG/ZEN/GTGAAGCTAGA/ |
| | 3IABkFQ/ (SEQ ID NO: 16) |
| | GACAGTGATAACGATGGTCCAG (SEQ ID NO: 17) |
| | ACCGTAAGATGGGTCAGTTTG (SEQ ID NO: 18) |
| PMA1 3′ | /56-FAM/CGTGTCTGG/ZEN/ATCTGGTCTATCGGTATCT/ |
| | 3IABkFQ/ (SEQ ID NO: 19) |
| | ACTGGACTGATATTGTTACTGTCG (SEQ ID NO: 20) |
| | CATCAATCTGTCAAAGGCTTCAG (SEQ ID NO: 21) |
| MDH2 | /56-FAM/AAAGTCAAA/ZEN/TCGGACCTTCCCAACCT/ |
| promoter | 3IABkFQ/ (SEQ ID NO: 22) |
| | TTTGTTTAGTGGCATTTTGTGTTG (SEQ ID NO: 23) |
| | CCTTAGATACCTTCCGTAGTTCC (SEQ ID NO: 24) |
| MDH2 5′ | /56-FAM/TGGTATCGG/ZEN/GCAGTCGTTATCGC/ |
| | 3IABkFQ/ (SEQ ID NO: 25) |
| | CTGGTATTGCAACTGAGCTTTC (SEQ ID NO: 26) |
| | ATTGCCATTTTAGGTGCTGC (SEQ ID NO: 27) |
| MDH2 3′ | /56-FAM/ATTATTGCC/ZEN/TCTGGTGGACGGTGC/ |
| | 3IABkFQ/ (SEQ ID NO: 28) |
| | AACTTCCCCATTGCTCCTG (SEQ ID NO: 29) |
| | GTTGCGTTCCATGTCGTTC (SEQ ID NO: 30) |
| HMG1 | /56-FAM/AGTGCGAAG/ZEN/AAAACGTAGTGCGAGT/ |
| promoter | 3IABkFQ/ (SEQ ID NO: 31) |
| | GAAGAGTTGGAAGACCTTCAGC (SEQ ID NO: 32) |
| | AGATCCTATAGCTAGTACGGACC (SEQ ID NO: 33) |
| HMG1 5′ | /56-FAM/TGAATGACG/ZEN/GATAGATAAGCGAATGCGG/ |
| | 3IABkFQ/ (SEQ ID NO: 34) |
| | CGGCGAAACGACCAATTC (SEQ ID NO: 35) |
| | GTGTTGGAGTCTTTATTTGGAGC (SEQ ID NO: 36) |
| HMG1 3′ | /56-FAM/AGGTTTTGT/Z EN/TGGTTCAGCAGGTTTCC/ |
| | 3IABkFQ/ (SEQ ID NO: 37) |
| | ACGATTTATATCAGTGGCGTCC (SEQ ID NO: 38) |
| | CGGCCATTTGGTTCAAAGTC (SEQ ID NO: 39) |
| ERG11 | /56-FAM/TCGTTAACT/ZEN/CGTGGAGATGCACAATAGG/ |
| promoter | 3IABkFQ/ (SEQ ID NO: 40) |
| | GCAGGAGACATCGATTTTATGC (SEQ ID NO: 41) |
| | GAGGCTTTTCGAATACATGCG (SEQ ID NO: 42) |
| ERG11 5′ | /56-FAM/TGGGCCAAT/ZEN/GGTAAAGCCAAGAAATG/ |
| | 3IABkFQ/ (SEQ ID NO: 43) |
| | TTGGAGAGGCATTGGAATACG (SEQ ID NO: 44) |
| | TCCTTTCTCAAAGAATATAGTAATTGCC |
| | (SEQ ID NO: 45) |
| ERG11 3′ | /56-FAM/ACCGTTCCA/ZEN/CCTCCTGACTTTACATCT/ |
| | 3IABkFQ/ (SEQ ID NO: 46) |
| | AAATGGCATTACCCAGAGGG (SEQ ID NO: 47) |
| | TTTGTTCTGGATTTCTCTTTTCCC (SEQ ID NO: 48) |
| HMG2 | /56-FAM/CCCTTTGGT/ZEN/ACTCCACCTCTTCAAGATG/ |
| promoter | 3IABkFQ/ (SEQ ID NO: 49) |
| | CACCTCAAGAACCTACCACAC (SEQ ID NO: 50) |
| | AGTGTCCTTACTATAACTTGCTCG (SEQ ID NO: 51) |
| HMG2 5′ | /56-FAM/CTCAGCTCT/ZEN/AAAGAAGCCGCCGAT/ |
| | 3IABkFQ/ (SEQ ID NO: 52) |
| | GGTCTCCTGTGTCTGATACATG (SEQ ID NO: 53) |
| | TCAAGGGAAGGCAAAGTCG (SEQ ID NO: 54) |
| HMG2 3′ | /56-FAM/CCGGTCACC/ZEN/TGGTACAAAGCCAT/ |
| | 3IABkFQ/ (SEQ ID NO: 55) |
| | GTGAACTGTCTCTGTGCTCC (SEQ ID NO: 56) |
| | GCCCTTTGTTACTTGGTTGTG (SEQ ID NO: 57) |
| YFR057W | /56-FAM/TGCCATGTA/ZEN/TGCAACTGCTCTACCA/ |
| upstream | 3IABkFQ/ (SEQ ID NO: 58) |
| inter- | ACACCCGTCTTTGATGCTAG (SEQ ID NO: 59) |
| genic | ATGTTCTCACGCTCTGTTCTAG (SEQ ID NO: 60) |
| YFR057W | /56-FAM/ACAAGGGAA/ZEN/CAATGAGCAGAGGGAAA/ |
| promoter | 3IABkFQ/ (SEQ ID NO: 61) |
| | CAAACAAGTAGGAATGCGAAAGG (SEQ ID NO: 62) |
| | AAGTGCTAAAGGAATCCCCAG (SEQ ID NO: 63) |
| YFR057W | /56-FAM/TGCTCGGCC/ZEN/AAGTCAAGCG/ |
| 5′ | 3IABkFQ/ (SEQ ID NO: 64) |
| | GCTTGGCGGTGTCTTTAATG (SEQ ID NO: 65) |
| | TGATATTTGGACCTACTAGTGTCTATAG |
| | (SEQ ID NO: 66) |
| YFR057W | /56-FAM/TGTCAAAGG/ZEN/AATTTTTTGCGCCTAGTGC/ |
| 3′ | 3IABkFQ/ (SEQ ID NO: 67) |
| | AAAGGATTTTAGCAACGACTTCG (SEQ ID NO: 68) |
| | TTGTCGAATCTTCCAGTAAATTTCTC (SEQ ID NO: 69) |
| YFR057W | /56-FAM/ACTTTCTGG/ZEN/AATAGCGTTCGGAATGTGT/ |
| 3′-UTR | 3IABkFQ/ (SEQ ID NO: 70) |
| | CACTCGTTAGGATCACGTTCG (SEQ ID NO: 71) |
| | TGTCGGAGAGTTAACAAGCG (SEQ ID NO: 72) |
| ARS504 | /56-FAM/TTGGTCTTC/ZEN/CCTACGCATTTCCCC/ |
| | 3IABkFQ/ (SEQ ID NO: 73) |
| | GCCGTTTCAATCTATCGTCATG (SEQ ID NO: 74) |
| | ATCCAGCTGACTCATTTCCTG (SEQ ID NO: 75) |

TABLE 3
Primers for ZipChIP analysis in A. thaliana

| | | Forward primer | Reverse primer |
|---|---|---|---|
| Gene | Region | sequence 5′-3′ | sequence 5′-3′ |
| ACT7 | 1 | TTTCGGCCCGTTTTA | GAGCCACCATGCTTC |
| | | TCGT | TCATT |
| | | (SEQ ID NO: 76) | (SEQ ID NO: 77) |
| ACT7 | 2 | TGGGGGTTGCTTGTT | AACAGCGAGAGATCG |
| | | ATGTG | ACAGA |
| | | (SEQ ID NO: 78) | (SEQ ID NO: 79) |
| ACT7 | 3 | AATACAGTGTCTGAT | GCGGAATTGGGATTT |
| | | CGGAGGAT | TTACCT |
| | | (SEQ ID NO: 80) | (SEQ ID NO: 81) |
| ACT7 | 4 | GAATAGTCCCTCGGG | TCGTGTTGGAATCAG |
| | | GTTGA | TTGATGA |
| | | (SEQ ID NO: 82) | (SEQ ID NO: 83) |
| MULE | 1 | CGAAGCGTTTGATCG | TTCGACTGTTTACCA |
| | | GGTAT | ACTGGA |
| | | (SEQ ID NO: 84) | (SEQ ID NO: 85) |
| MULE | 2 | CTTGGAACTTCCCGA | CACCACTTCACAAAC |
| | | GAGCTT | CCACCTA |
| | | (SEQ ID NO: 86) | (SEQ ID NO: 87) |
| MULE | 3 | CCCATGATTGACAGT | AGGGCTATGAGTTGC |
| | | CGTTGA | CAAGA |
| | | (SEQ ID NO: 88) | (SEQ ID NO: 89) |
| MULE | 4 | ACACCTTCGAGACTT | TGTACAGCGGCGTTA |
| | | CCCTT | AAACA |
| | | (SEQ ID NO: 90) | (SEQ ID NO: 91) |

TABLE 4
ChIP analysis of H3K4me3 comparing ZipChIP to standard ChIP

| | | Fold change | |
|---|---|---|---|
| Procedure | Gene location | relative to set1Δ | P value |
| ZipChIP | PYK1 promoter | 44.29 +/− 15.94 | 0.053 |
| ZipChIP | PYK1 5′ | 216.79 +/− 33.37 | 0.003 |
| ZipChIP | PYK1 3′ | 23.88 +/− 5.72 | 0.016 |
| Standard | PYK1 promoter | 47.83 +/− 14.28 | 0.031 |
| Standard | PYK1 5′ | 161.06 +/− 37.43 | 0.013 |
| Standard | PYK1 3′ | 26.30 +/− 6.41 | 0.017 |
| ZipChIP | PMA1 promoter | 50.90 +/− 14.12 | 0.024 |
| ZipChIP | PMA1 5′ | 107.70 +/− 28.40 | 0.020 |
| ZipChIP | PMA1 3′ | 15.17 +/− 5.56 | 0.063 |
| Standard | PMA1 promoter | 49.25 +/− 11.84 | 0.015 |
| Standard | PMA1 5′ | 91.88 +/− 17.18 | 0.006 |
| Standard | PMA1 3′ | 10.37 +/− 1.80 | 0.007 |
| ZipChIP | YFR057W promoter | 7.34 +/− 2.58 | 0.070 |
| ZipChIP | YFR057W 5′ | 5.72 +/− 1.65 | 0.046 |
| ZipChIP | YFR057W 3′ | 7.62 +/− 3.74 | 0.151 |
| Standard | YF057W promoter | 6.26 +/− 1.57 | 0.028 |
| Standard | YFR057W 5′ | 5.79 +/− 1.42 | 0.028 |
| Standard | YFR057W/ 3′ | 6.94 +/− 1.55 | 0.019 |

TABLE 5
ChIP analysis of H3K4me3 in a set1Δ strain comparing ZipChIP to
standard ChIP

| | | Fold change relative to |
|---|---|---|
| Procedure | Gene location | individual gene promoter |
| ZipChIP | PYK1 promoter | |
| ZipChIP | PYK1 5′ | 1.51 +/− 0.24 |
| ZipChIP | PYK1 3′ | 1.47 +/− 0.95 |
| Standard | PYK1 promoter | |
| Standard | PYK1 5′ | 1.64 +/− 0.70 |
| Standard | PYK1 3′ | 1.16 +/− 0.09 |
| ZipChIP | PMA1 promoter | |
| ZipChIP | PMA1 5′ | 2.08 +/− 0.32 |
| ZipChIP | PMA1 3′ | 1.90 +/− 0.58 |
| Standard | PMA1 promoter | |
| Standard | PMA1 5′ | 1.41 +/− 0.51 |
| Standard | PMA1 3′ | −1.41 +/− 0.49 |
| ZipChIP | YFR057W promoter | |
| ZipChIP | YFR057W 5′ | −1.06 +/− 0.09 |
| ZipChIP | YFR057W 3′ | 1.03 +/− 0.22 |
| Standard | YF057W promoter | |
| Standard | YFR057W 5′ | −1.04 +/− 0.13 |
| Standard | YFR057W 3′ | −1.05 +/− 0.20 |

TABLE 6
ChIP analysis of H3K4me3 in a WT strain comparing ZipChIP to
standard ChIP

| | | Fold change relative to |
|---|---|---|
| Procedure | Gene location | individual gene promoter |
| ZipChIP | PYK1 promoter | |
| ZipChIP | PYK1 5′ | 9.39 +/− 2.14 |
| ZipChIP | PYK1 3′ | −1.21 +/− 0.06 |
| Standard | PYK1 promoter | |
| Standard | PYK1 5′ | 5.04 +/− 0.56 |
| Standard | PYK1 3′ | −1.64 +/− 0.06 |
| ZipChIP | PMA1 promoter | |
| ZipChIP | PMA1 5′ | 4.46 +/− 0.65 |
| ZipChIP | PMA1 3′ | −2.24 +/− 0.02 |
| Standard | PMA1 promoter | |
| Standard | PMA1 5′ | 2.25 +/− 0.50 |
| Standard | PMA1 3′ | −6.92 +/− 0.10 |
| ZipChIP | YFR057W promoter | |
| ZipChIP | YFR057W 5′ | −1.33 +/− 0.02 |
| ZipChIP | YFR057W 3′ | −1.06 +/− 0.27 |
| Standard | YF057W promoter | |
| Standard | YFR057W 5′ | −1.12 +/− 0.11 |
| Standard | YFR057W 3′ | −1.55 +/− 0.26 |

TABLE 7
ChIP analysis of H3K4me3 comparing ZipChIP to standard ChIP

| | | Fold change relative to |
|---|---|---|
| Procedure | Gene location | YFR057W promoter |
| ZipChIP | PYK1 promoter | 6.58 +/− 1.10 |
| ZipChIP | PYK1 5′ | 57.06 +/− 4.13 |
| ZipChIP | PYK1 3′ | 5.31 +/− 0.50 |
| Standard | PYK1 promoter | 8.66 +/− 0.64 |
| Standard | PYK1 5′ | 43.04 +/− 0.83 |
| Standard | PYK1 3′ | 5.36 +/− 0.85 |
| ZipChIP | PMA1 promoter | 5.89 +/− 0.52 |
| ZipChIP | PMA1 5′ | 26.69 +/− 5.83 |
| ZipChIP | PMA1 3′ | 2.62 +/− 0.28 |
| Standard | PMA1 promoter | 8.19 +/− 1.30 |
| Standard | PMA1 5′ | 17.23 +/− 1.87 |
| Standard | PMA1 3′ | 0.94 +/− 0.48 |

TABLE 8
ChIP analysis of Jhd2-3xFLAG comparing ZipChIP to standard

| | | Fold change | |
|---|---|---|---|
| Procedure | Gene location | relative to WT | P value |
| ZipChIP | PYK1 promoter | 11.37 +/− 2.51 | 0.015 |
| ZipChIP | PYK1 5′ | 11.45 +/− 0.33 | 6.21 × 10⁶ |
| ZipChIP | PYK1 3′ | 13.50 +/− 3.25 | 0.018 |
| Standard | PYK1 promoter | 1.17 +/− 0.15 | 0.330 |
| Standard | PYK1 5′ | −1.03 +/− 0.18 | 0.874 |
| Standard | PYK1 3′ | −1.02 +/− 0.11 | 0.851 |
| ZipChIP | PMA1 promoter | 10.33 +/− 4.85 | 0.127 |
| ZipChIP | PMA1 5′ | 8.40 +/− 1.03 | 0.002 |
| ZipChIP | PMA1 3′ | 9.68 +/− 0.62 | 0.0002 |
| Standard | PMA1 promoter | 1.05 +/− 0.09 | 0.651 |
| Standard | PMA1 5′ | 1.03 +/− 0.19 | 0.889 |
| Standard | PMA1 3′ | −1.03 +/− 0.14 | 0.809 |
| ZipChIP | ARS504 | 2.54 +/− 0.43 | 0.024 |

A. thaliana Material and Growth Conditions:

Seeds used in these studies were obtained from wild-type or transgenic plants grown in parallel in an AR75 incubator (Percival Scientific) under 24 hr of illumination. Seeds were allowed to dry for at least a month on the plant prior to collection. No other treatment was applied (i.e. stratification) prior to use of the seeds. Plants were incubated on synthetic media and grown in a CU36L5 incubator (Percival Scientific) under 24 hr of illumination. Whole plants were collected 14 days post imbibition for ChIP analysis.

Isolating Nuclei and Generating Soluble Chromatin from A. thaliana:

The nuclei extraction and generation of soluble chromatin from A. thaliana were performed as previously described. Tissue was immersed in crosslinking solution (0.002% Silwet; 10 mM Tris-HCl, pH8.0; 0.44 M Sucrose; 1% formaldehyde) under house vacuum. After 10 min, 1/15 volume of 2 M glycine was added, and the vacuum was reapplied for another 5 min. The tissues were washed and flash-frozen in liquid nitrogen. The tissue was ground into a fine powder and resuspended in 10 mL of cold Extraction Buffer 1 (0.4 M sucrose; 10 mM Tris-HCl, pH 8.0; 10 mM MgCl₂) with protease inhibitors (lx protease inhibitor cocktail; Sigma P9599). The following steps were performed at 4° C. or on ice unless otherwise indicated. The lysate was filtered through one layer of cheesecloth (VWR 21910-107). After filtering, the cheesecloth was rinsed with another 10 mL of cold Extraction Buffer 1. All 20 mL of Extraction Buffer 1 containing tissue samples were then filtered through one layer of Miracloth (Calbiochem 475855) and centrifuged at 3,000×g for 20 min. The pellet was resuspended in 1.5 mL of Extraction Buffer 2 (0.25 M sucrose; 10 mM Tris-HCl, pH 8.0; 10 mM MgCl₂; 1% Triton X-100) with protease inhibitors and centrifuged at 2,000×g for 10 min. Each pellet was resuspended in another 1 mL of Extraction Buffer 2 with protease inhibitors and centrifuged at 2,000×g. Each pellet was then resuspended in 600 μl of Extraction Buffer 3 (1.7 M sucrose; 10 mM Tris-HCl, pH 8.0; 2 mM MgCl₂; 0.15% Triton X-100) with protease inhibitors, layered on top of another 600 μL of Extraction Buffer 3 and centrifuged (16,000×g, 1 hour). Pellets from each tube were resuspended in 750 μL of HEPES buffer (50 mM HEPES-KOH, pH 7.5; 140 mM NaCl; 1% Triton X-100; 0.1% Na deoxycholate; 1 mM EDTA) with protease inhibitors and the chromatin was sheared into ˜250-750 bp fragments by sonication (Misonix sonicator: Power 6, 10 seconds, 6 times with 20 second rest). The tubes were then centrifuged (16,000×g, 10 min), and the supernatant was transferred to a new tube.

A. thaliana ZipChIP Protocol:

1 μL of antibody (anti-H3, Abcam ab1791; anti-H3K4me3, Millipore 07-473), or no antibody was conjugated to 10 μL magnetic beads (Dynabeads, Life Technologies 10004D; equilibrated with PBS-T, 50% slurry) for about 30 min. Before performing the immunoprecipitation, the soluble lysate was precleared by rocking with 5 μL of Protein-G magnetic beads (Dynabeads, Life Technologies 10004D; equilibrated with HEPES buffer, 50% slurry) at 4° C. for 30 min. 200 μL of the precleared supernatant was added to the beads conjugated with the respective antibodies and incubated for 2 hours. The beads were washed sequentially with 1 ml of each of the following: aFA-140 wash buffer (aFA-140 buffer refers to the FA-140 buffer used for Arabidopsis thaliana; 50 mM HEPES-KOH, pH 7.5; 140 mM NaCl; 1% Triton X-100; 1 mM EDTA), aFA-500 wash buffer (as HEPES buffer, but with 500 mM NaCl and 0.1% Na deoxycholate), and 1×PBS-T. For the A. thaliana ZipChIP, on-bead analysis was not used due to the use of SYBR Green Universal Master Mix for detection of PCR amplification. The immunocomplexes were eluted by heating at 100° C. for 10 min. The samples were cooled to room temperature. The tubes were then spun at 12,000×g for 1 min and the supernatant removed to a new tube.

A. thaliana Real-Time PCR and Statistical Analysis:

qRT-PCR was performed using the StepOnePlus Real-Time PCR System (Life Technologies). 4 μL of DNA was used for each of the three technical repeats for each of the three biological repeats. The DNA was amplified using SYBR Green Universal Master Mix (Life Technologies) and the primers described in Table 3. The data were analyzed using the ΔΔCt method for PKL-cMYC and the ΔCt method for H3K4me3. The mean relative fold changes or ΔCt values, s.e.m., and statistical significance values are found in Tables 14 and 15.

TABLE 9
Fold difference of Jhd2-3xFLAG levels at PYK1 and PMA1
compared to ARS504

| | Fold change relative |
|---|---|
| Gene location | to ARS504 locus |
| ARS504 | 1 |
| PYK1 promoter | 4.48 |
| PYK1 5′ | 4.51 |
| PYK1 3′ | 5.32 |
| ARS504 | 1 |
| PMA1 promoter | 4.07 |
| PMA1 5′ | 3.31 |
| PMA1 3′ | 3.82 |

TABLE 10
ChIP analysis of Rph1-3xFLAG comparing ZipChIP to standard ChIP

| | | Fold change | |
|---|---|---|---|
| Procedure | Gene location | relative to WT | P value |
| ZipChIP | PYK1 promoter | 9.26 +/− 1.52 | 0.006 |
| ZipChIP | PYK1 5′ | 16.96 +/− 2.27 | 0.002 |
| ZipChIP | PYK1 3′ | 19.55 +/− 2.80 | 0.003 |
| Standard | PYK1 promoter | 1.15 +/− 0.24 | 0.566 |
| Standard | PYK1 5′ | 1.35 +/− 0.24 | 0.219 |
| Standard | PYK1 3′ | 1.32 +/− 0.29 | 0.335 |
| ZipChIP | PMA1 promoter | 15.47 +/− 3.72 | 0.018 |
| ZipChIP | PMA1 5′ | 24.92 +/− 3.00 | 0.001 |
| ZipChIP | PMA1 3′ | 20.53 +/− 1.77 | 0.0004 |
| Standard | PMA1 promoter | 1.30 +/− 0.38 | 0.476 |
| Standard | PMA1 5′ | 1.28 +/− 0.25 | 0.316 |
| Standard | PMA1 3′ | 1.30 +/− 0.13 | 0.079 |
| ZipChIP | ARS504 | 17.41 +/− 5.88 | 0.049 |

TABLE 11
ZipChIP analysis of Sir2

| | | Fold change |
|---|---|---|
| Gene location | Region | relative to No Ab |
| YFR057W upstream intergenic | 1 | 1.45 +/− 0.17 |
| YFR057W promoter | 2 | 1.26 +/− 0.73 |
| YFR057W 5′ | 3 | 4.04 +/− 0.28 |
| YFR057W 3′ | 4 | 6.11 +/− 1.85 |
| YFR057W 3′ UTR | 5 | 7.44 +/− 0.82 |
| PYK1 3′ | NA | 1.81 +/− 0.36 |
| PMA1 3′ | NA | 1.59 +/− 0.18 |

TABLE 12
ZipChIP analysis of H3k4me1

| | Fold change | |
|---|---|---|
| Gene location | relative to set1Δ | P value |
| PYK1 promoter | 4.76 +/− 1.36 | 0.051 |
| PYK1 5′ | 4.87 +/− 0.90 | 0.012 |
| PYK1 3′ | 24.73 +/− 5.55 | 0.013 |
| MDH2 promoter | 3.43 +/− 0.18 | 0.0002 |
| MDH2 5′ | 8.91 +/− 0.75 | 0.0005 |
| MDH2 3′ | 31.66 +/− 4.72 | 0.003 |
| PMA1 promoter | 5.12 +/− 1.48 | 0.050 |
| PMA1 5′ | 8.60 +/− 1.21 | 0.003 |
| PMA1 3′ | 5.31 +/− 0.32 | 0.003 |
| HMG1 promoter | 8.99 +/− 2.43 | 0.030 |
| HMG1 5′ | 6.56 +/− 1.63 | 0.027 |
| HMG1 3′ | 13.07 +/− 2.66 | 0.011 |
| ERG11 promoter | 9.48 +/− 1.13 | 0.0003 |
| ERG11 5′ | 9.16 +/− 1.80 | 0.004 |
| ERG11 3′ | 24.33 +/− 6.65 | 0.013 |
| HMG2 promoter | 8.79 +/− 1.77 | 0.005 |
| HMG2 5′ | 18.67 +/− 4.00 | 0.005 |
| HMG2 3′ | 14.61 +/− 3.39 | 0.007 |

TABLE 13
ZipChIP analysis of H3K4me1 in a rad6Δ and bre1Δ

| rad6Δ | | bre1Δ | |
|---|---|---|---|
| | Fold change | | Fold change |
| Gene location | relative to set1Δ | Gene location | relative to set1Δ |
| PYK1 | 4.23 +/− 2.39 | PYK1 promoter | 2.61 +/− 1.08 |
| promoter | | | |
| PYK1 5′ | 4.11 +/− 0.27 | PYK1 5′ | 5.11 +/− 0.78 |
| PYK1 3′ | 1.47 +/− 0.21 | PYK1 3′ | 1.89 +/− 0.29 |
| MDH2 | 2.34 +/− 0.61 | MDH2 promoter | 2.21 +/− 0.66 |
| promoter | | | |
| MDH2 5′ | 3.47 +/− 0.34 | MDH2 5′ | 6.02 +/− 2.65 |
| MDH2 3′ | 4.56 +/− 2.76 | MDH2 3′ | 5.04 +/− 2.85 |
| PMA1 | 2.33 +/− 0.28 | PMA1 promoter | 1.45 +/− 0.34 |
| promoter | | | |
| PMA1 5′ | 2.63 +/− 0.69 | PMA1 5′ | 3.55 +/− 1.06 |
| PMA1 3′ | 1.99 +/− 0.98 | PMA1 3′ | 1.39 +/− 1.06 |
| HMG1 | 4.14 +/− 1.24 | HMG1 promoter | 5.76 +/− 3.37 |
| promoter | | | |
| HMG1 5′ | 9.14 +/− 1.91 | HMG1 5′ | 10.87 +/− 2.88 |
| HMG1 3′ | 6.33 +/− 2.85 | HMG1 3′ | 5.64 +/− 2.45 |

TABLE 14
ZipChIP analysis of H3K4me3 in A. thaliana

| Gene | Region | ΔCt |
|---|---|---|
| ACT7 | 1 | 0.14 +/− 0.01 |
| ACT7 | 2 | 0.65 +/− 0.08 |
| ACT7 | 3 | 0.16 +/− 0.03 |
| ACT7 | 4 | 0.15 +/− 0.01 |
| MULE | 1 | 0.06 +/− 0.01 |
| MULE | 2 | 0.06 +/− 0.01 |
| MULE | 3 | 0.06 +/− 0.01 |
| MULE | 4 | 0.11 +/− 0.03 |

TABLE 15
ZipChIP analysis of MYC-PKL in A. thaliana

| Gene | Region | Fold change relative to WT | P value |
|---|---|---|---|
| ACT7 | 1 | 4.58 +/− 0.40 | 0.039 |
| ACT7 | 2 | 5.62 +/− 1.79 | 0.019 |
| ACT7 | 3 | 3.65 +/− 0.75 | 0.050 |
| ACT7 | 4 | 8.00 +/− 2.76 | 0.015 |
| MULE | 1 | 1.83 +/− 0.29 | 0.051 |
| MULE | 2 | 1.83 +/− 0.51 | 0.082 |
| MULE | 3 | 1.87 +/− 0.14 | 0.072 |
| MULE | 4 | 2.48 +/− 0.52 | 0.087 |

### FURTHER REPRESENTATIVE EXAMPLES

### Example 1

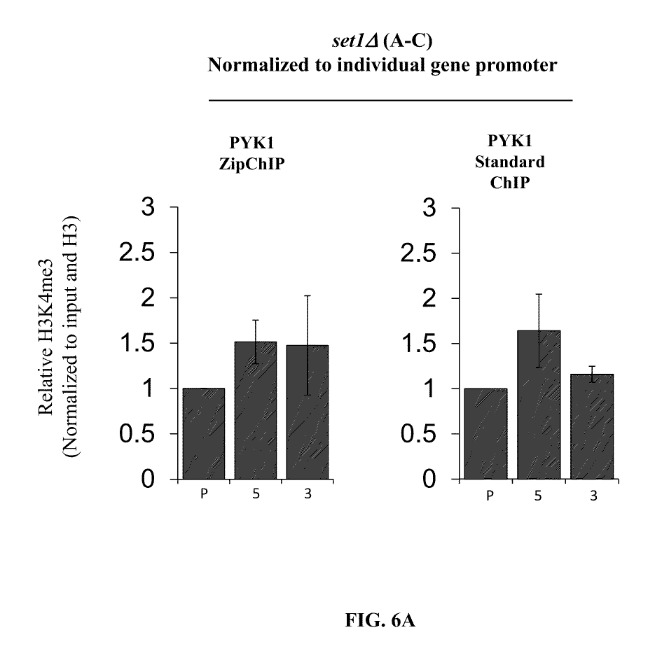

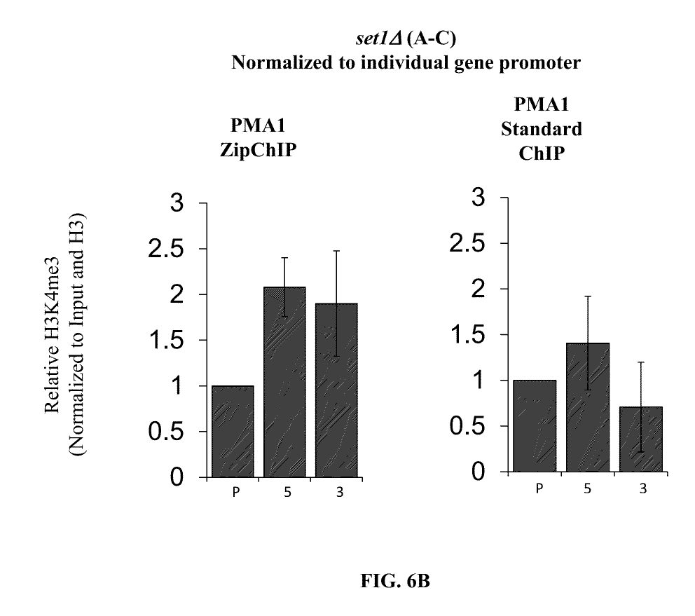

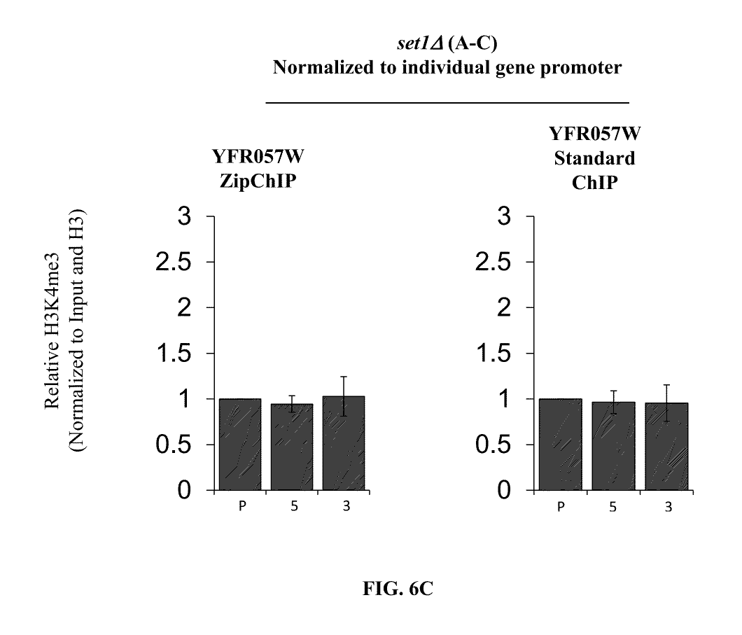

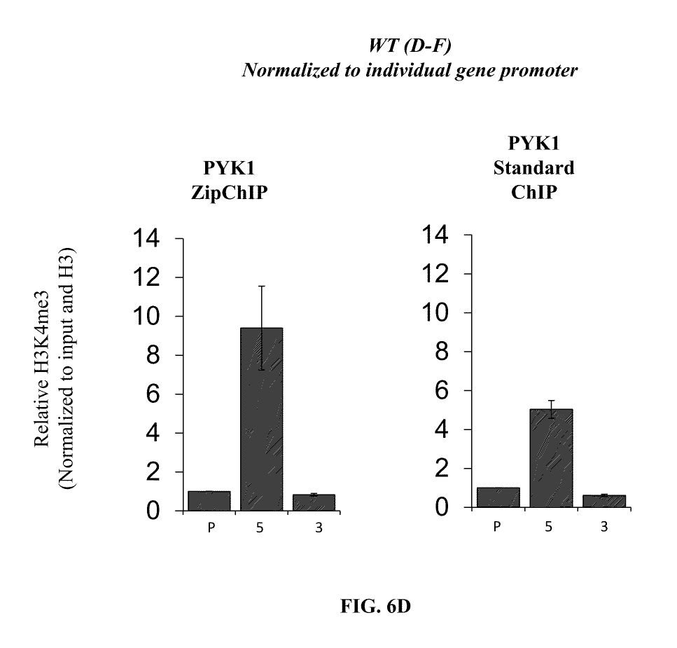

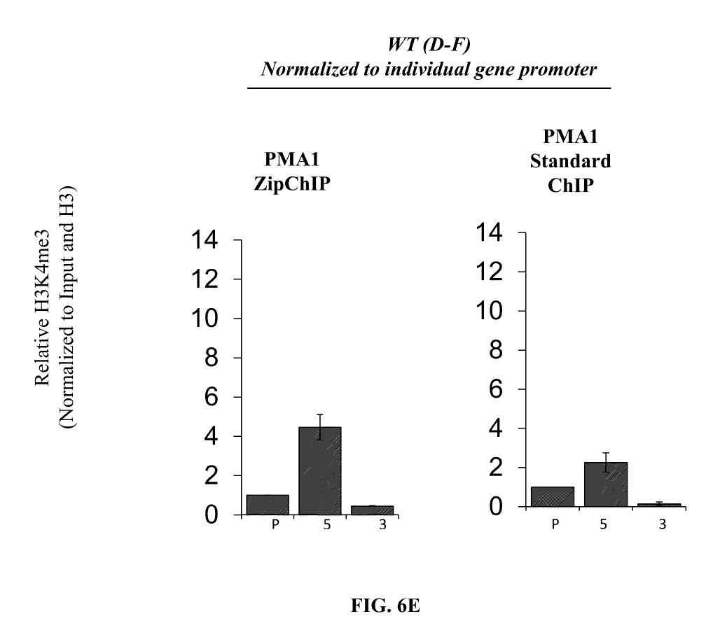

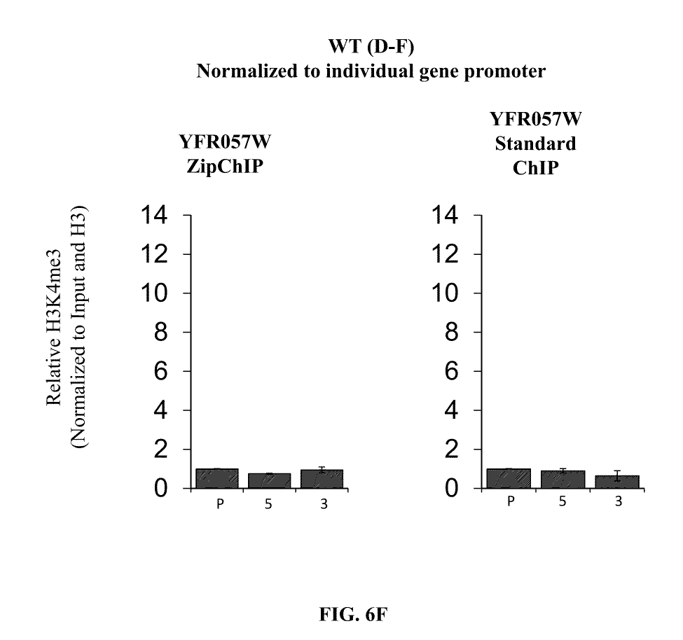

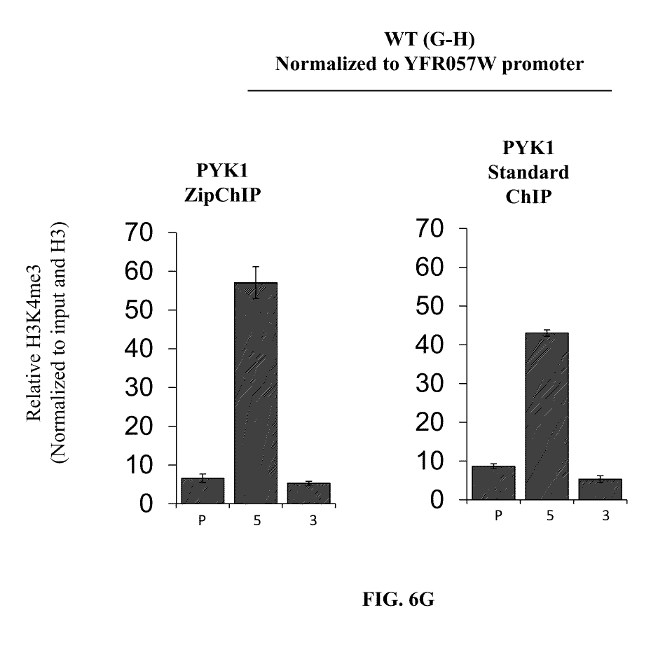

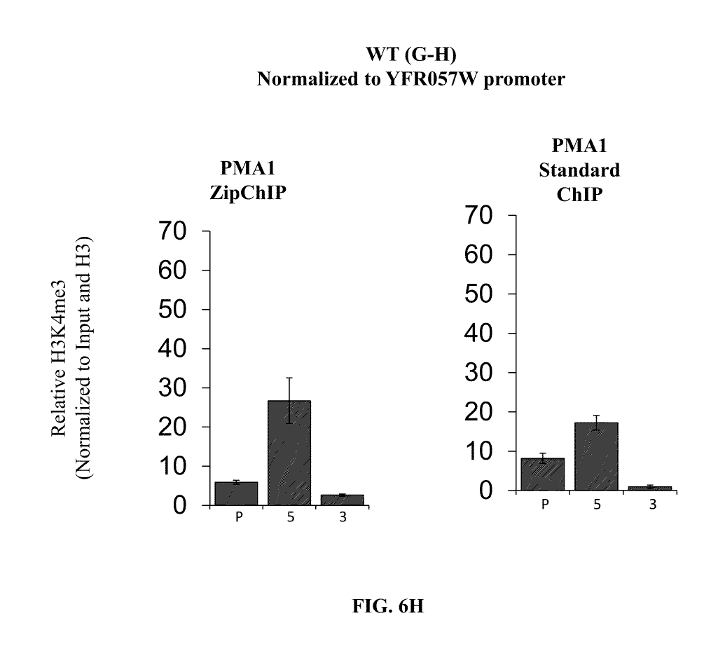

Verification of ZipChIP through analysis of the pattern of H3K4 trimethylation across actively transcribed genes. Many studies have characterized a specific pattern of H3K4 methylation across the open-reading frame (ORF) of actively transcribed genes, with H3K4 trimethylation (H3K4me3) being enriched at the 5′-ORF and transcriptional start site, H3K4 dimethylation (H3K4me2) localized in the middle of the gene, and H3K4 monomethylation (H3K4me1) enriched at the 3′-ORF. To establish that ZipChIP analysis is a reliable and robust method, ZipChIP was compared to a standard “long” ChIP method using an H3K4me3-specific antibody. The general scheme of the ZipChIP protocol is depicted in FIG. 1A. Yeast cells were grown to log phase and cross-linked with formaldehyde. After cell lysis and sonication, chromatin was purified. For the DNA input control, 6.25% of soluble chromatin was removed and processed with a standard PCR clean up kit (Qiagen). During the chromatin preparation, the H3K4me3 antibody (Millipore) was conjugated to Protein-G magnetic beads (DynaBeads, Life Technologies) for 30 minutes. The soluble chromatin was then immunoprecipitated with the antibody-conjugated magnetic beads for 2 hours. After immunoprecipitation, the beads were washed three times for 5 minutes each and resuspended in water. Referring now to FIGS. 1A-D, ChIP analysis was performed on BY4741 wild-type (WT) and set1Δ strains using antibodies specific for H3K4me3 and histone H3. Input and histone H3 were used for normalization. ChIP analysis is relative to the H3K4me3 signal for the set1Δ strain. Three biological replicates with three technical repeats were used for all ZipChIP and standard ChIP analysis. The error bars represent the standard error of the mean. Referring now to FIGS. 1B and 1C, the H3K4me3 ZipChIP sample was then analyzed using qRT-PCR at the promoter, 5′-ORF, and 3′-ORF of two constitutively active genes, PYK1 and PMA1. As a negative control, H3K4me3 ZipChIP was performed in a yeast strain lacking the only known H3K4 histone methyltransferase, Set1. In the set1Δ strain, H3K4 mono-, di-, and trimethylation are abolished. A standard ChIP method was also used to analyze the H3K4me3 pattern across these two genes in both a wild-type (WT) strain and a set1Δ strain as shown in FIG. 1B, FIG. 1C, and FIG. 6. Both methods showed the characteristic peak of H3K4me3 at the 5′-ORF of both genes. Furthermore, the signal of the H3K4me3 detected was very similar between the two methods when normalizing to the H3K4 trimethylation signal for a set1Δ strain (FIG. 1B and FIG. 1C), when normalizing to the signal H3K4 trimethylation signal at the promoter of the individual gene in the WT strain (FIG. 6D and FIG. 6E), and normalizing to the H3K4 trimethylation signal of the sub-telomere gene YFR057W (FIG. 6G and FIG. 61I). This indicates that the ZipChIP method can be used to detect H3K4me3 in about 6 hours after formaldehyde cross-linking compared to the 3 days using standard ChIP. As a control to show that the methylation detected at the two active genes PMA1 and PYK1 was specific, a silent sub-telomere gene, YFR057W, was analyzed for H3K4me3. Both methods showed very low levels of H3K4me3 at YFR057W when compared to PYK1 and PMA1, as expected due to the correlation between H3K4me3 and active transcription (FIG. 1D and FIG. 6C). Referring now to FIGS. 6A-6C, to show H3K4me3 ZipChIP background levels in a set1Δ strain, the background signal for each gene was normalized to the promoter of each gene. Data generated in FIG. 1 were also normalized to their respective gene promoter as shown in FIGS. 6D-6H. In addition, PYK1 and PMA1 were also normalized with respect to the YFR057W promoter as shown in FIGS. 6G-6H.

### Example 2

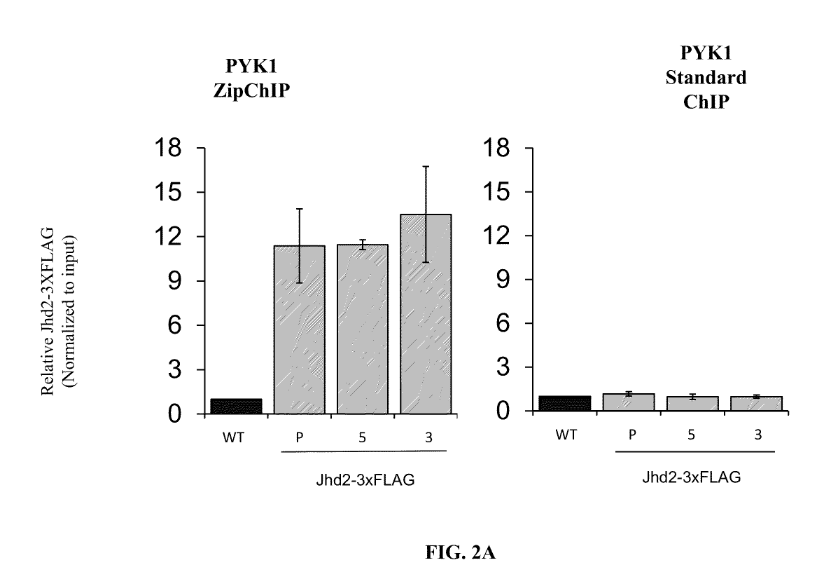

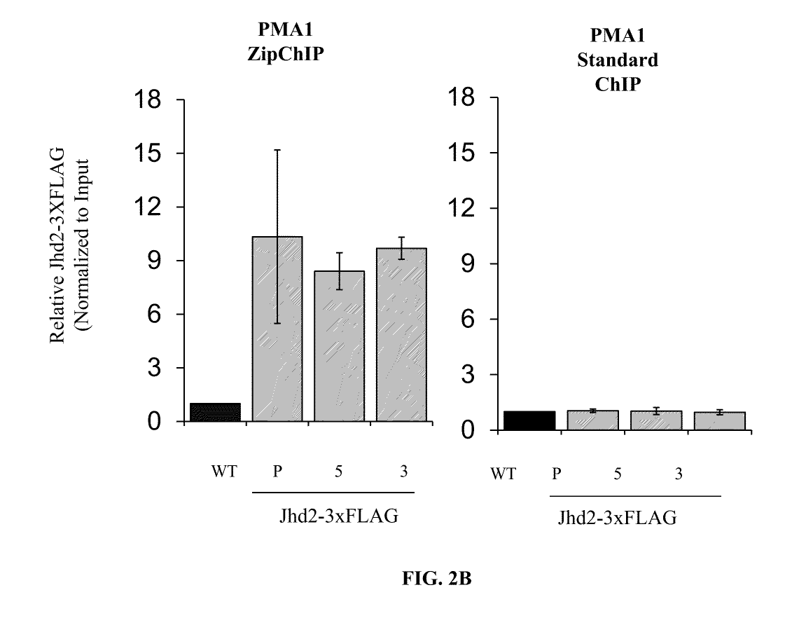

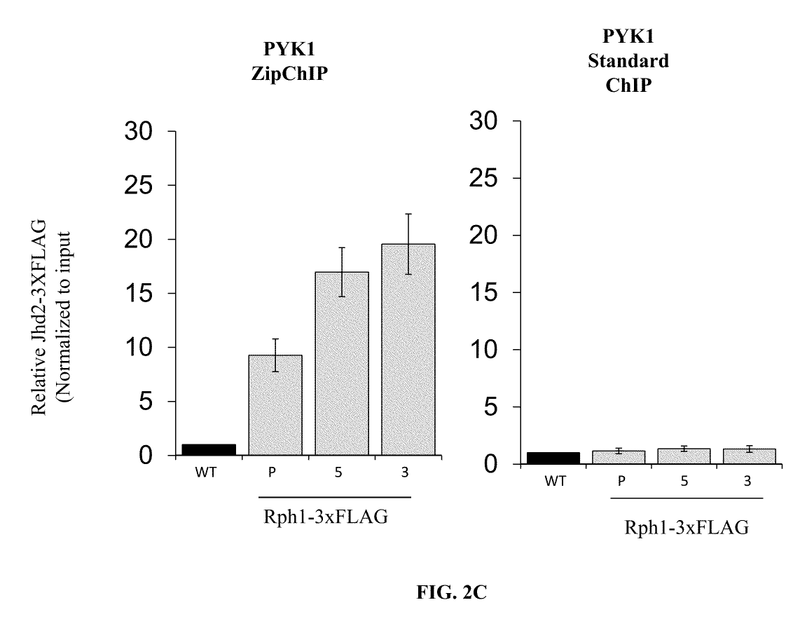

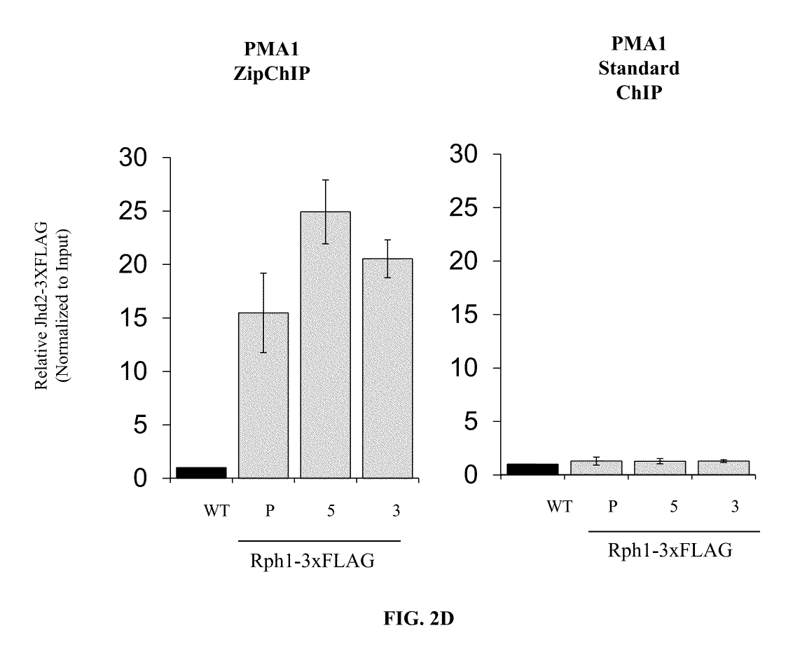

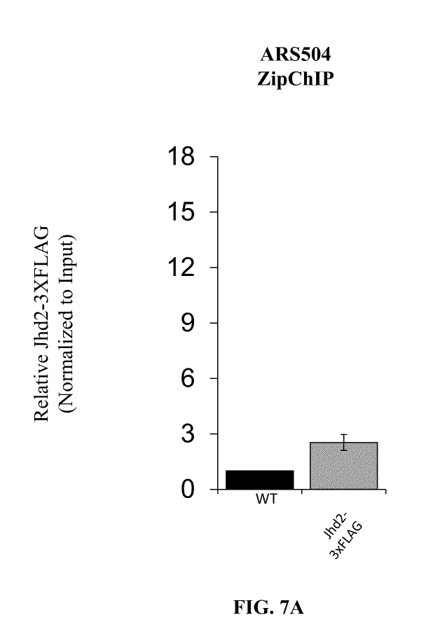

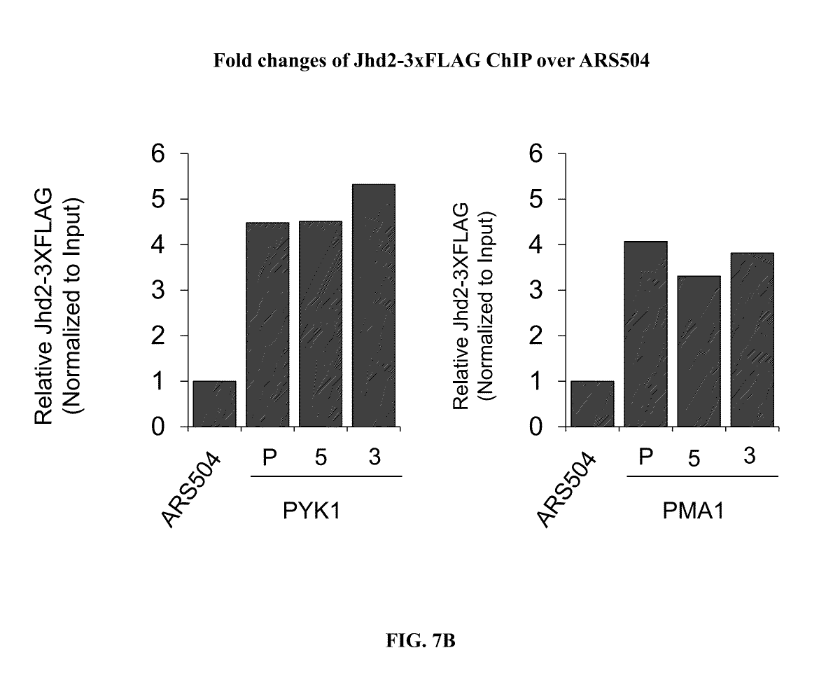

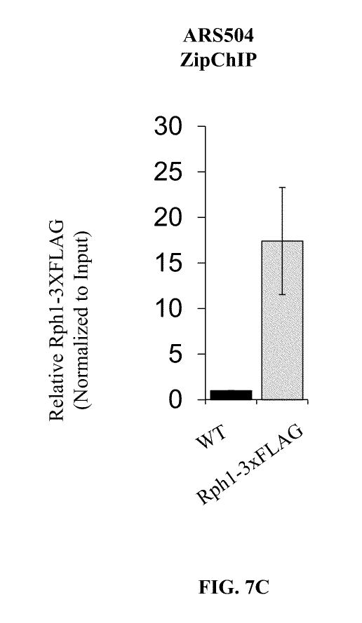

Histone demethylases, Jhd2 and Rph1, associate with the promoter, 5′-ORF, and 3-ORF of actively transcribed genes. Studies have described protein factors that have been difficult or nearly impossible to detect using the standard ChIP protocol. For example, it has been reported that the yeast H3K4 histone demethylase Jhd2 cannot be analyzed using standard ChIP methods. However, using the ZipChIP protocol, there was the ability to readily detect Jhd2 and the H3K36 histone demethylase Rph1 interacting with the promoter, 5′-ORF, and 3′-ORF of two actively transcribed genes, PYK1 and PMA1, with a signal that was significantly over the untagged Jhd2 and untagged Rph1 background signal, respectively as shown in FIGS. 2A-D. In contrast, a signal was not observed over background for either Jhd2 or Rph1 using the standard ChIP method as shown in FIGS. 2A-D. Referring now to FIGS. 2A-D, an α-FLAG antibody was used for immunoprecipitation of both Jhd2-3×FLAG and Rph1-3×FLAG for both ChIP methods. Input was used for normalization, and ChIP analysis is relative to a WT untagged control. Three biological replicates with three technical repeats were used for all ZipChIP and standard ChIP analysis. The error bars represent the standard error of the mean. To determine if the signal observed for Jhd2 at PYK1 and PMA1 a specific, a locus with low levels of Jhd2 was analysed. The levels of H3K4 demethylase Jhd2 were about 3.3-5.3 fold higher at the actively transcribed genes compared to ARS504 when comparing FIG. 2A, FIG. 2B, FIG. 7A, and FIG. 7B. This indicates that there Jhd2 is enriched at PYK1 and PMA1 compared to ARS504. Low levels of protein at ARS504 was specific for Jhd2 as Rph1 was detected at this locus at similar levels to what was detected at PYK1 and PMA1 as shown in FIG. 7C. The ability to analyze chromatin-associated proteins, which have been difficult to detect using standard ChIP, makes the ZipChIP method a significant and important tool for future work characterizing the association of these and other chromatin-associated proteins to specific loci.

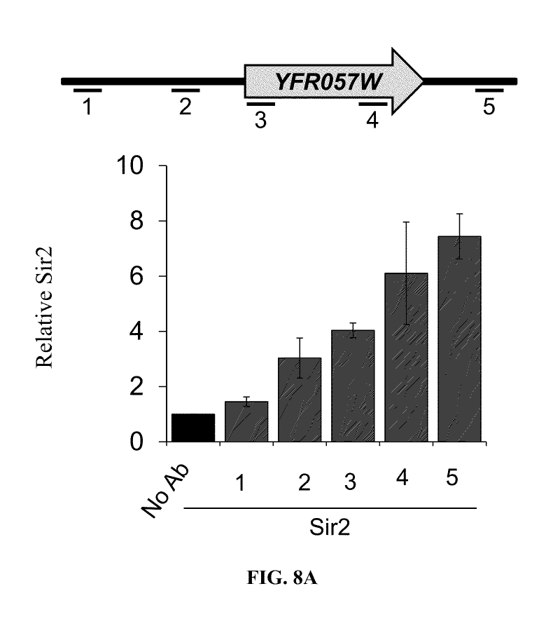

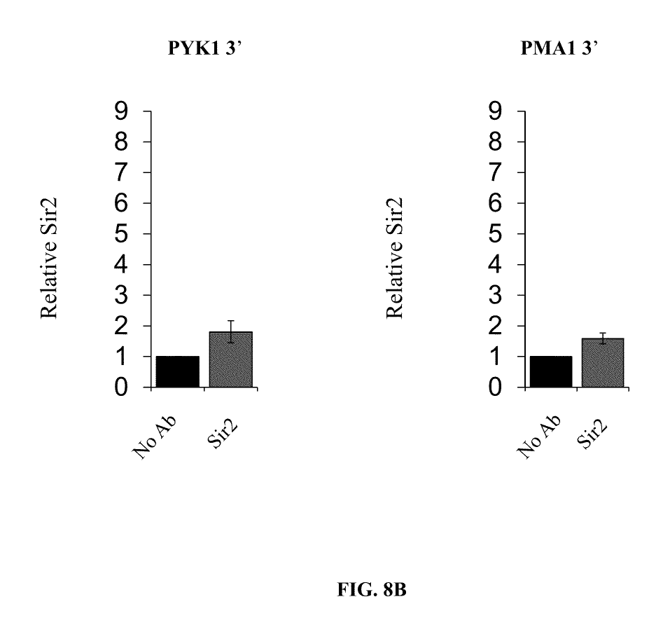

To further establish that ZipChIP can show specific enrichment of proteins and to determine if ZipChIP can be used to determine enrichment of a chromatin associated protein using a protein specific antibody, levels of the histone deacetylase Sir2 was analyzed. This protein has been shown to localize at the silent genes including the sub-telomere gene YFR057W. Therefore, a Sir2 specific antibody was used to assess Sir2 levels at YFR057W and at two actively transcribed genes PYK1 and PMA1 as shown in FIG. 8A and FIG. 8B. Using ZipChIP, Sir2 enrichment was observed at YFR057W 3′-ORF and 3′-UTR (untranslated region) as shown in FIG. 8A, and as expected low levels of Sir2 were observed at the 3′-ORFs of PYK1 and PMA1 as shown in FIG. 8B. This further illustrates that ZipChIP is specific and works with antibodies that recognize epitope tagged proteins or endogenous proteins.

### Example 3

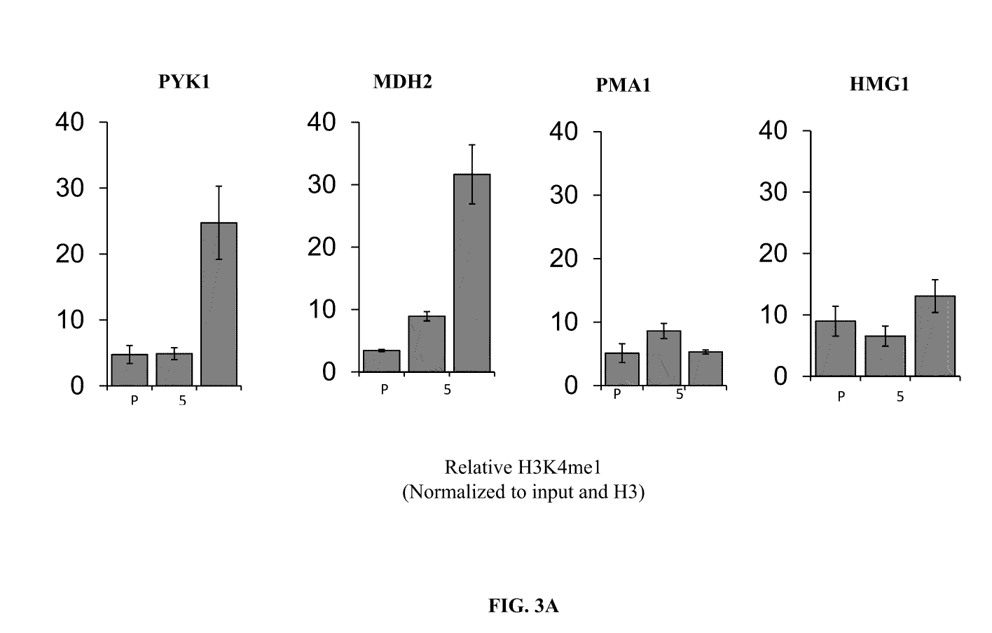

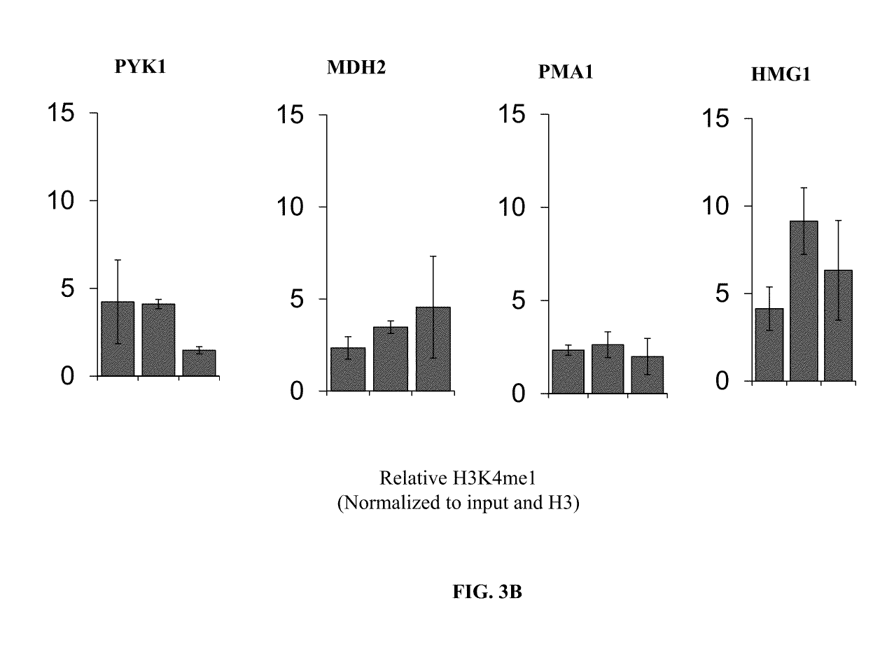

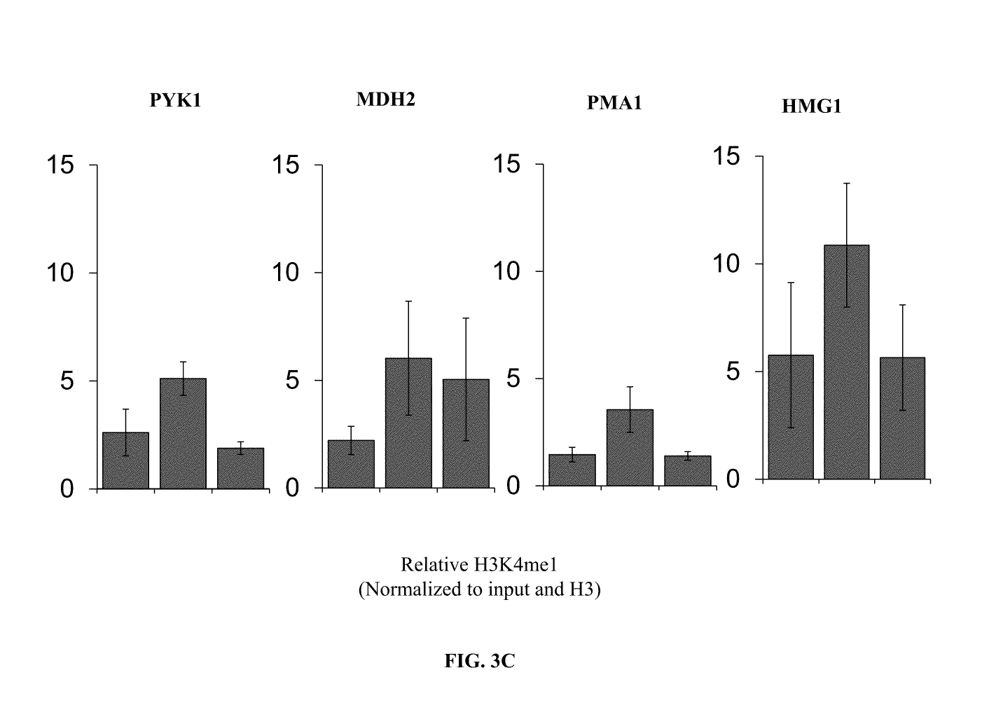

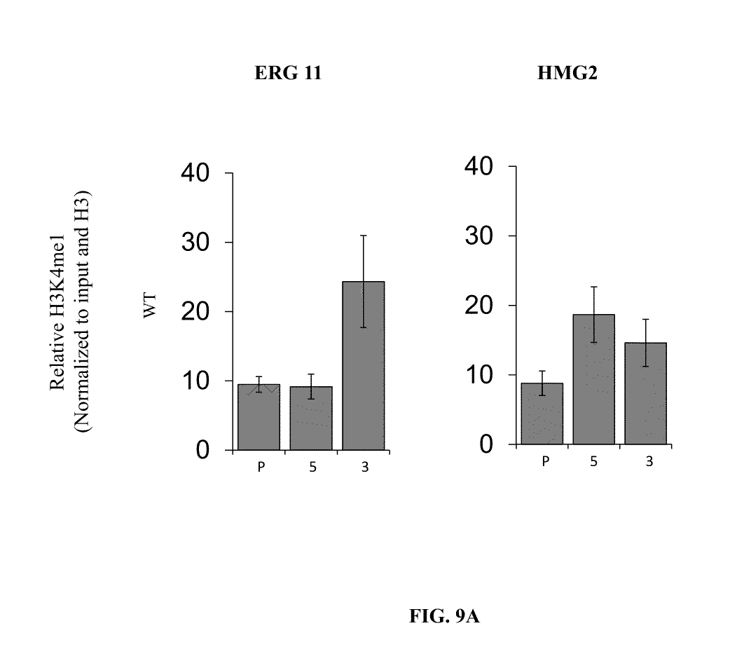

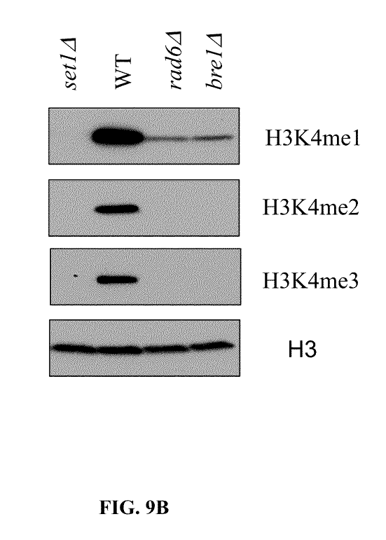

H3K4 monomethylation is enriched at the 3′-ORF for a subset of actively transcribed genes and this enrichment is lost in both rad6Δ and bre1Δ strains. Although genome-wide approaches can provide valuable initial information there are limitations with every technology. For example, reports of false positives due to sample bias and of low number of reads from certain targets necessitate validation and follow-up analysis of genome-wide localization data. As previously mentioned H3K4me1 has been shown to be enriched at the 3′-ORF of actively transcribed genes. However, this particular histone modification has been challenging to study using standard ChIP in yeast, so its biological role is not fully understood. To determine if ZipChIP can be used to detect H3K4me1 in genomic regions, both WT and set1Δ strains were cross-linked and soluble chromatin was isolated from these strains. The Protein-G magnetic beads were conjugated with a commercially available H3K4me1 antibody (Active Motif) and used in the immunoprecipitation of soluble chromatin. Referring now to FIGS. 3A-C, input and histone H3 were used for normalization. ZipChIP analysis is relative to the H3K4me1 signal for the set1Δ strain. Three biological replicates with three technical repeats were used for ZipChIP analysis. The error bars represent the standard error of the mean. Due to the lesser amount of this modification, the amount of soluble chromatin used in the immunoprecipitation was increased 1200% (300 μL) compared to that used for H3K4me3 ChIP (25 μL). After the ZipChIP method was completed, qRT-PCR analysis was used to analyze H3K4me1 levels at the promoter, 5′-ORF, and 3′-ORF of four active genes that according to ChIP-chip data have H3K4me1 3′-ORF enrichment (PYK1, PMA1, MDH2, HMG1, and HMG2). Interestingly, the H3K4me1 ZipChIP results differ from that of the ChIP-chip data. Relative to the background signal of a set1Δ strain, PYK1, MDH2, and ERG11 had an enrichment of H3K4me1 at the 3′-ORF, consistent with enrichment pattern previously reported as shown in FIG. 3A and FIG. 9A. However, though H3K4me1 was detected at the active genes PMA1, HMG1, and HMG2, a 3′-ORF enrichment was not observed, differing from the previously reported ChIP-chip data as shown in FIG. 3A and FIG. 9A.

While not wishing to be bound by theory, due to the ability to detect significant levels of H3K4 monomethylation at actively transcribed genes using ZipChIP, it was questioned how other PTMs could affect the enrichment pattern of H3K4 monomethylation. The histone crosstalk between H3K4 methylation and H2B K123 monoubiquitination has been thoroughly studied, but still not completely understood. Multiple studies have shown that H2B monoubiquitination is necessary for the establishment of H3K4 di- and trimethylation. Global H3K4 di- and trimethylation were lost when RAD6 or BRE1, the gene encoding the E2 ubiquitin-conjugating enzyme and the gene encoding the E3 ubiquitin ligase responsible for H2B monoubiquitination, respectively, was deleted as shown in FIG. 9B. However, though global H3K4 monomethylation is reduced it is still detectable through Western blot analysis as shown in FIG. 9B. This suggests that though Rad6 and Bre1 affect the overall levels H3K4 monomethylation, some H3K4 monomethylation can be established even with their absence. How exactly Rad6 and Bre1 affect H3K4 monomethylation across a gene and if they play a role in the observed 3′ enrichment of H3K4 monomethylation is largely unknown. One study showed that in a rad6Δ an increase in H3K4 monomethylation was observed at the 5′-ORF of two actively transcribed genes, but also that H3 levels at the locus were higher in a rad6Δ compared to WT. This study also stated that no H3K4 monomethylation was observed at the 3′-ORF of the genes analyzed in a rad6Δ strain. Another study also looked at H3K4 monomethylation across a gene in both a rad6Δ and a bre1Δ strain but reported that no change in signal was observed compared to WT.

To resolve these differences, ZipChIP was used to analyse the levels of H3K4 monomethylation in rad6Δ and bre1Δ strains across PYK1 and MDH2, the two genes observed to have a 3′-ORF enrichment of monomethylation in a WT strain (FIG. 3A). The ChIP analysis revealed that the 3′-ORF H3K4 monomethylation enrichment was lost at PYK1 and MDH2 in both rad6Δ and bre1Δ strains as shown in FIGS. 3B and 3C. Interestingly, H3K4 monomethylation signal at the 3′-ORF of PYK1 was significantly lower (p-value <0.05) compared to the 5′-ORF signal of PYK1 in both rad6Δ and bre1Δ strains as shown in FIGS. 3B and 3C. Furthermore, PMA1 and HMG1 were analyzed in a rad6Δ strain and bre1Δ strain, and as observed in the WT strain, levels of H3K4 monomethylation were similar across the promoter, 5′-ORF, and 3′-ORF as shown in FIGS. 3B and 3C. Taken together, ZipChIP was able to identify that there are two distinct patterns of H3K4 monomethylation and that H2B ubiquitination is necessary for H3K4 monomethylation enrichment at the 3′ open-reading frame of some but not all genes.

### Example 4

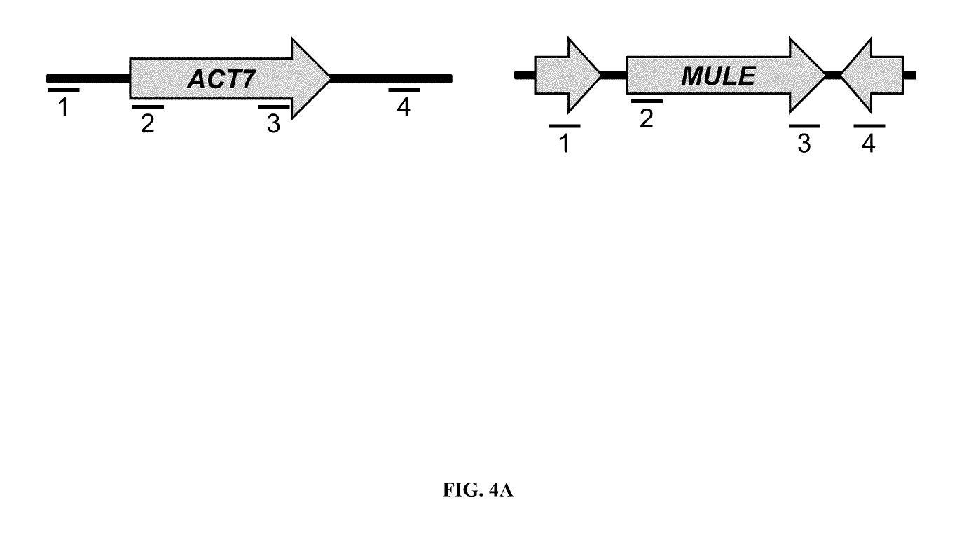

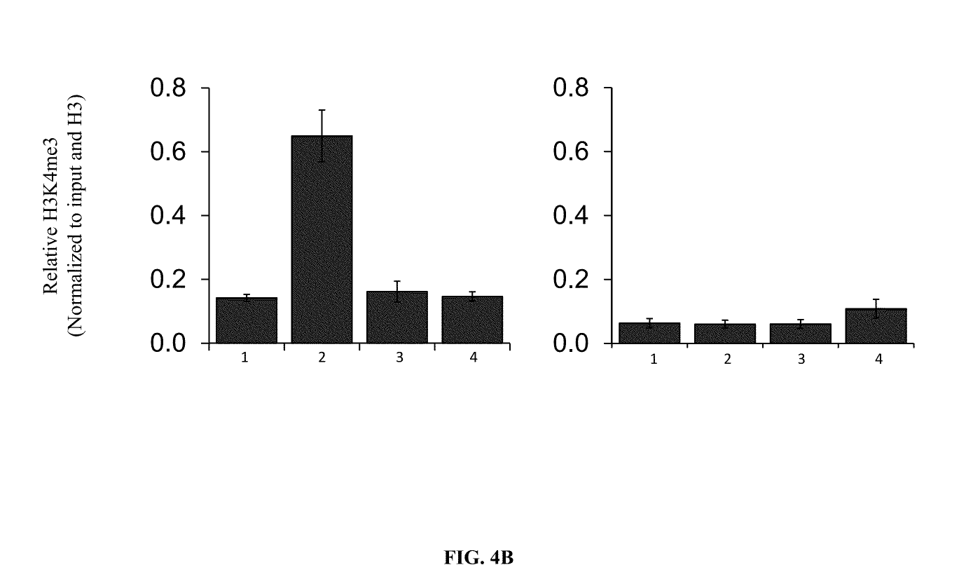

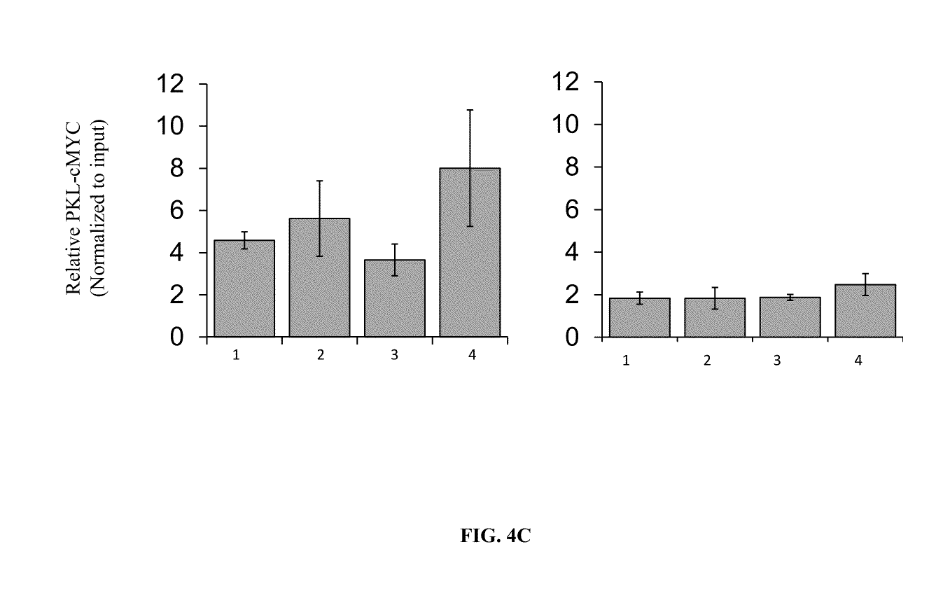

The ZipChIP method can be applied to study the relationship between chromatin modifications and remodelers in Arabidopsis thaliana. In this representative example, A. thaliana chromatin remodeler PKL localization does not correlate with H3K4 trimethylation enrichment at the active gene ACT7. Additionally, ZipChIP was tested for use with more complex genomes. PICKLE (PKL) is an ATP-dependent remodeler that promotes H3K27me3, a mark associated with transcriptional repression. Interestingly, PKL not only associates with H3K27me3 enriched genes but is also found at actively transcribed genes, such as the promoter of ACTT. The association of PKL with the promoter of ACT7 introduces the possibility of a new role for PKL and raises the prospect that PKL may co-localize with an epigenetic modification associated with actively transcribed genes such as H3K4me3. Using ZipChIP to compare both PKL-cMYC and H3K4me3 localization at ACT7 and a heterochromatic locus, MULE, it allows a user to quickly screen through multiple regions to determine if there is any correlation between PKL localization and H3K4me3 enrichment as shown in FIG. 4A. In agreement with published genome-wide analyses, H3K4me3 was observed to be enriched at the 5′-ORF of ACT7, and no H3K4me3 enrichment was observed at the heterochromatic gene MULE as shown in FIG. 4B. It was observed that although PKL preferentially associated with ACT7 relative to MULE, as reported previously, the association of PKL at ACTT was relatively constant in each region examined and did not correlate with H3K4me3 enrichment when comparing FIG. 4B and FIG. 4C. Therefore, using the ZipChIP method the localization of both H3K4me3 and PKL could be quickly scanned across genes to show that even though PKL preferentially interacts with chromatin at actively transcribed genes, there is no correlation between PKL interaction and the enrichment of H3K4 trimethylation.

Referring now to FIG. 5, the ZipChIP method was used to analyze histones and histone modifications at the HMG-CoA reductase (HMGCR) gene at exon1 from human LNCaP prostate cancer cells. This showed that ZipChIP method worked in a mammalian cell and both histones and histone H3K4 trimethylation was detected at the HMGCR gene at exon1.

Those skilled in the art will recognize that numerous modifications can be made to the specific implementations described above. The implementations should not be limited to the particular limitations described. Other implementations may be possible.

While the inventions have been illustrated and described in detail in the drawings and foregoing description, the same is to be considered as illustrative and not restrictive in character, it being understood that only certain embodiments have been shown and described and that all changes and modifications that come within the spirit of the invention are desired to be protected.

What is claimed is:

1. A method of analyzing chromatin, comprising;
conjugating a portion of solubilized chromatin with at least one antibody, wherein the antibody is attached to a water insoluble support, to form a chromatin water insoluble support complex;
immunoprecipitating the chromatin water insoluble support complex, to form a immunopreciptated chromatin water insoluble support complex; and
analyzing the immunopreciptated chromatin water insoluble support complex for the presence of chromatin.

2. The method according to claim 1, wherein the water insoluble support complex, is at least one structure selected from the group consisting of: a metal bead, an agarose bead, a sepharose, or a chip surface.

3. The method according to claim 1, wherein the solubilized chromatin is extracted from at least one cell selected from the group of prokaryotic cells, eukaryotic cell, bacteria cells, plant cells, fungal cells, and animal cells.

4. The method according to claim 1, wherein the solubilized chromatin is extracted from at least one cell using a SB-140 lysis buffer.

5. The method according to claim 1, the antibody is capable or preferentially binding to at least one post translationally modified protein.

6. The method according to claim 1, the antibody is capable or preferentially binding to at least one methylated protein.

7. The method according to claim 1, further including the step of washing the immunoprecipitating the chromatin water insoluble support complex.

8. The method according to claim 1, wherein the analytical step includes the use of DNA primers.

9. A method of analyzing nucleic acids, comprising:
conjugating a portion of solubilized chromatin with at least one antibody, wherein the antibody is attached to a water insoluble support, to form a chromatin water insoluble support complex;
immunoprecipitating the chromatin water insoluble support complex, to form a immunopreciptated chromatin water insoluble support complex; and
analyzing the immunopreciptated chromatin water insoluble support complex for the presence DNA and/or RNA.

10. The method according to claim 9, wherein the water insoluble support complex, is at least one structure selected from the group consisting of: a metal bead, an agarose bead, a sepharose, or a chip surface.

11. The method according to claim 9, wherein the solubilized chromatin is extracted from at least one cell selected from the group of prokaryotic cells, eukaryotic cell, bacteria cells, plant cells, fungal cells, and animal cells.

12. The method according to claim 9, wherein the solubilized chromatin is extracted from at least one cell using a SB-140 lysis buffer.

13. The method according to claim 9, the antibody is capable or preferentially binding to at least one post translationally modified protein.

14. The method according to claim 9, the antibody is capable or preferentially binding to at least one methylated protein.

15. The method according to claim 9, further including the step of washing the immunoprecipitating the chromatin water insoluble support complex.

16. The method according to claim 1, wherein the analytical step includes the use of nucleic acid primers.

17. A kit, comprising:
at least one first antibody attached to at least one water insoluble support, wherein the antibody preferentially binds to at least one protein present in a portion of chromatin to form a chromatin antibody complex; and
at least one lysate buffer, wherein said buffer is suitable for forming water soluble chromatin.

18. The kit according to claim 17, further including a wash buffer, wherein the wash buffer is suitable for washing said chromatin antibody complex.

19. The kit according to claim 17, further including at least one primer, that at least one primer binds to at least one portion of a nucleic acid present in said chromatin antibody complex.

20. The kit according to claim 17, wherein the water insoluble support is at least structure selected from the group consisting of metal, agarose, or sepharose beads.
